SEQUENCE LISTING (4 sequences)

SEQ ID NO: 1 - protein, 938 aa, Mus musculus
  MISC_FEATURE (1)..(938): Accession NP_032195
MSTMHLLTFALLFSCSFARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYNWNHIILLVSDDHEGRAAQKRLETLLEERESKAEKVLQFDPGTKNVTALLMEARDLEARVIILSASEDDAATVYRAAAMLNMTGSGYVWLVGEREISGNALRYAPDGIIGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTMSDGTCKEEFTVNGDPVKKVICTGPNDTSPGSPRHTVPQCCYGFCVDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQDRKSGRAEPDPKKKATFRAITSTLASSFKRRRSSKDTSTGGGRGALQNQKDTVLPRRAIEREEGQLQLCSRHRES

SEQ ID NO: 2 - DNA, 2817 bp, Mus musculus
  misc_feature (1)..(2817): Accession CCDS15764.1
atgagcaccatgcacctgctgacattcgccctgcttttctcctgctccttcgcccgcgctgcctgcgaccccaagattgtcaacatcggcgcggtgctgagcacgcgcaagcacgagcagatgttccgcgaggcagtaaaccaggccaataagcgacacggctcttggaagatacagctcaacgccacttctgtcacccacaagcccaacgccatacagatggccctgtcagtgtgtgaggacctcatctctagccaggtctacgctatcctagttagtcacccgcctactcccaacgaccacttcactcccacccctgtctcctacacagctggcttctacagaatccccgtcctggggctgactacccgaatgtccatctactctgacaagagcatccacctgagcttccttcgcaccgtaccaccctactcccaccagtccagcgtctggtttgagatgatgcgcgtctacaactggaaccatatcatcctgctggtcagcgatgaccacgagggccgggcagcgcagaagcgcctggagacgttgctggaggagcgtgagtccaaggcagagaaggtgctgcagtttgacccaggaaccaagaatgtgacggctctgctgatggaagcccgggacctggaagcccgggtcatcatcctttctgcaagcgaggacgacgctgccaccgtataccgcgcagccgcgatgctgaacatgactggctctgggtacgtgtggctcgtcggggagcgcgagatctctgggaatgccctgcgctacgctcctgacggcatcatcggacttcagctaatcaacggcaagaacgagtcggcccacatcagtgacgctgtgggcgtggtggcacaggcagtccacgagctcctagaaaaggagaacatcactgatccaccgcggggttgcgtgggcaacaccaacatctggaagacaggaccactgttcaagagggtgctgatgtcttccaagtatgcagatggagtgactggccgtgtggaattcaatgaggatggggaccggaagtttgccaactatagtatcatgaacctgcagaaccgcaagctggtgcaagtgggcatctacaatggtacccatgtcatcccaaatgacaggaagatcatctggccaggaggagagacagagaagcctcgaggataccagatgtccaccagactaaagatagtgacaatccaccaagaacccttcgtgtatgtcaagcccacaatgagtgatggcacatgcaaagaggagttcacagtcaatggtgaccctgtcaagaaggtgatctgtacggggcctaatgacacatccccaggaagcccacgtcacacagtgccccagtgctgttatggcttctgcgttgacctgctcatcaagctggcacggaccatgaattttacctacgaggtgcaccttgtggcagatggcaagtttggcacacaggagcgggtaaacaacagcaacaaaaaggagtggaacggaatgatgggagagctgctcagtggtcaagcagacatgatcgtggctccactgaccattaacaatgagcgtgcgcagtacatagagttctccaagcccttcaagtaccagggcctgaccattctggtcaagaaggagatccctcggagcacactggactcattcatgcagccctttcagagcacactgtggctgctggtggggctgtcagttcatgtggtggccgtgatgctgtacctgctggaccgcttcagtccctttggccgatttaaggtgaacagcgaggaggaggaggaggatgcactgaccctgtcctctgccatgtggttttcctggggcgtcctgctcaactctggcattggggaaggtgccccccggagtttctctgctcgtatcctaggcatggtgtgggctggttttgccatgatcatcgtggcttcctacactgccaacctggcagccttcctggtgctggataggcctgaggagcgcatcacaggcatcaatgaccccaggctcagaaacccctcagacaagttcatctatgcaactgtaaaacagagctctgtggatatctacttccggaggcaggtggagttgagcaccatgtaccggcacatggagaagcacaattatgagagtgcagctgaggccatccaggctgtgcgggacaacaagctccatgccttcatctgggactcagctgtgctggagtttgaggcttcacagaagtgcgatctggtgaccacgggtgagctgttcttccgctccggctttggcatcggcatgcgcaaggacagcccctggaagcaaaatgtgtccctgtccatactcaagtcccatgagaatggcttcatggaagacctggataagacatgggttcggtatcaagaatgtgactcccgcagcaatgcccctgccaccctcacttttgagaacatggcaggggtcttcatgctggtggctggaggcatcgtagctgggatcttcctcattttcatcgagatcgcctacaagcgacacaaggatgcccgtaggaagcagatgcagctggcttttgcagccgtgaacgtgtggaggaagaacctgcaggatagaaagagtggtagagcagagcccgaccctaaaaagaaagccacatttagggctatcacctccaccctggcctccagcttcaagagacgtaggtcctccaaagacacgagcaccgggggtggacgcggcgctttgcaaaaccaaaaagacacagtgctgccgcgacgcgctattgagagggaggagggccagctgcagctgtgttcccgtcatagggagagctga

SEQ ID NO: 3 - protein, 1464 aa, Mus musculus
  MISC_FEATURE (1)..(1464): Accession NP_032196
MGRLGYWTLLVLPALLVWHGPAQNAAAEKGTPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQTEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTKFNQRGVEDALVSLKTGKLDAFIYDAAVLNYKAGRDEGCKLVTIGSGYIFATTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISNMSNMNSSRMDSPKRAADFIQRGSLIVDMVSDKGNLIYSDNRSFQGKDSIFGENMNELQTFVANRHKDSLSNYVFQGQHPLTLNESNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSEVERTYVKTKASSPRDKIYTIDGEKEPSFHLDPPQFIENIVLPENVDFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEAYQQDWSQNNALQFQKNKLKINRQHSYDNILDKPREIDLSRPSRSISLKDRERLLEGNLYGSLFSVPSSKLLGNKSSLFPQGLEDSKRSKSLLPDHTSDNPFLHTYGDDQRLVIGRCPSDPYKHSLPSQAVNDSYLRSSLRSTASYCSRDSRGHSDVYISEHVMPYAANKNNMYSTPRVLNSCSNRRVYKKMPSIESDV

SEQ ID NO: 4 - DNA, 4395 bp, Mus musculus
  misc_feature (1)..(4395): Accession CCDS27943.1
atgggcagactgggctactggaccttgctggtattgccggcccttctggtctggcacggtccggcgcagaacgcggcggcggagaagggtactccagcgctgaacattgcggtgctgctgggtcacagccacgacgtgacagaacgcgaacttcgaaatctgtggggcccggagcaggcaaccggcttgcccctggatgtgaacgtggtggcgttattgatgaaccgcactgaccctaagagcctcatcacgcatgtgtgcgacctcatgtccggggcgcgcatccatggcttggtgtttggagatgatacggaccaagaggctgtggctcagatgctggattttatctcctcacagactttcatccccatcttgggcattcatgggggtgcatctatgatcatggctgacaaggatccgacatccacgttcttccagtttggagcttccatccagcagcaagccacggtcatgctgaagatcatgcaggactatgactggcatgtcttctccttggtcaccaccatcttccctggctacagagacttcatcagcttcatcaagacaacagtggacaacagctttgtgggctgggatatgcagaacgtgatcacactggacacctcttttgaggacgccaagacacaggtccagctgaagaagatccactcctctgtcatcctgctctactgctccaaggatgaggctgtcctcatcctgagcgaggctcgctctcttggcctcaccggctacgatttcttctggattgtccccagtttggtctccgggaacacagagctcatccccaaagagtttccatcgggtctcatttcagtctcttacgacgactgggactacagtctggaggcaagagtgagagacggtcttgggatcttaaccactgccgcatcttccatgttggagaaattctcctacattcccgaggccaaggccagctgctacgggcagacagagaagccggagaccccgctacacactctgcaccaatttatggtcaatgtgacttgggatggcaaggacttgtccttcactgaggaaggctatcaggtgcaccccaggcttgtggtgatcgtgctgaataaggaccgggaatgggaaaaggtgggcaagtgggagaatcagactctgagcctgcggcatgctgtgtggccaaggtataagtccttttctgactgtgagccagatgacaaccacctcagcattgtcaccttggaggaagcccccttcgtcatcgtagaagacatagacccactgactgagacctgcgtcaggaacacggtaccctgtcggaagtttgtcaagatcaacaattcaaccaacgaagggatgaatgtgaagaagtgctgcaaggggttctgcatcgacatccttaagaaactgtccagaactgtaaagttcacctatgacctctacctggtgaccaatgggaagcatgggaaaaaggttaacaatgtgtggaatggaatgataggcgaagtggtctatcaacgagcagttatggccgtgggctccctcaccatcaatgaggagcgttcagaagtggtggacttctccgtgccctttgtggagacaggaatcagtgtcatggtctccaggagtaatggcactgtttccccttctgctttccttgaacccttcagcgcctctgtctgggtgatgatgttcgtgatgctgctcattgtctctgccattgctgtcttcgtttttgaatacttcagtcctgttggatacaacagaaacttagccaaagggaaagctccccatgggccttcttttaccattggaaaagctatatggctcctctggggcctggtcttcaacaattctgtgcccgtccagaatcctaaaggcacaaccagcaagataatggtatcagtgtgggccttctttgccgtcatcttccttgcaagttacacagccaacctggctgccttcatgatccaggaggagtttgtggaccaagtgactggcctcagtgacaagaagttccagagacctcatgactattctccgcctttccgatttgggacagtacccaatggaagtacagaaaggaatattcgtaacaactacccctatatgcaccagtacatgaccaaattcaaccagaggggcgtagaggatgccttggtcagcttgaaaactgggaagttggacgctttcatctatgacgcagctgtcttgaactacaaggccgggagggatgaaggctgtaaactggtgaccattgggagcgggtacatctttgccaccacaggctatggaattgctctgcagaagggctcaccctggaagaggcagattgacctcgctctgctccagtttgttggtgacggtgagatggaagagctggagacactgtggcttacgggcatctgccacaacgagaagaatgaggtgatgagcagccagctggacatcgacaacatggcaggagttttctacatgctggctgcagctatggccctcagcctcatcaccttcatctgggagcacctcttctactggaagctgcgcttctgcttcacaggcgtgtgttctgaccggcccgggctgctcttctccatcagcaggggcatctacagttgcatccatggagtgcacattgaagaaaagaagaagtctccagacttcaatctgactgggtcacagagcaacatgctaaagcttctccgctcagctaaaaacatctccaacatgtccaacatgaactcctcgcgaatggactcacccaaaagagctgctgacttcatccaaagaggctcacttattgtggacatggtttcagacaagggaaatttgatatactcagataacaggtcctttcaagggaaggacagtatatttggagaaaacatgaatgaactgcaaacatttgtggccaacaggcacaaggatagtctcagtaactatgtgtttcagggacagcatcctctcactctcaatgagtccaaccccaacacagtggaggtggctgtcagcactgaatccaaagggaactcccgaccccggcagctttggaagaaatccatggagtctctacgccaggattctctaaaccagaacccagtctcccagagggatgagaagactgcagagaataggacccactccctaaagagtcctaggtatcttccagaagaggtagcccattctgacatttctgaaacctcaagccgggccacatgccacagggagccagataataataagaaccacaagaccaaggataacttcaaaaggtcaatggcctctaaataccccaaggactgtagtgaggttgaacgtacctacgtgaaaaccaaagcaagttctcccagggataagatctacaccatcgatggtgagaaggagcccagcttccacttagatcctccacagttcattgaaaacatagtcttgcctgagaatgtggacttcccagatacctaccaagatcacaatgagaatttccgcaagggggactccacactgcccatgaacaggaacccactacacaatgaagatgggcttcccaacaatgaccagtataaactctatgccaagcactttaccttgaaagacaagggttccccacatagtgagggcagtgatcgatatcggcagaactccacgcattgcagaagctgcctctcaaacctgcccacctactcaggccactttaccatgagatctcctttcaagtgtgatgcctgtctgcggatggggaacctctatgacattgatgaagaccagatgcttcaggagacaggcaacccagctactcgtgaggaggcctaccagcaggactggtcacagaacaacgccctccagttccagaagaacaagctaaagattaatcgacagcactcctatgataacattctcgacaaacccagggagatagaccttagcaggccctctcgtagcataagcctcaaggacagggaaaggctactggagggcaacttatacgggagcctgttcagtgtcccctcaagcaaactcttggggaacaaaagctcccttttcccccaaggtctggaggacagcaagaggagcaaatctctcttgccagaccatacctctgataatcctttcctccacacgtacggggatgaccaacgcttagttattgggagatgtccctcggacccttacaaacactcattgccatcacaggcagtaaatgacagctatcttcggtcatccttgaggtcaacagcatcatattgctccagggacagtcggggccacagtgatgtgtatatttcagagcatgttatgccttatgctgcaaataagaataacatgtactctacccccagggttttaaattcctgcagcaatagacgtgtgtacaagaaaatgcctagtattgaatctgatgtctaa

(19) United States
(12) Patent Application Publication
(10) Pub. No.: US 2011/0072525 A1
(43) Pub. Date: Mar. 24, 2011

(54) COMPOSITIONS AND METHODS FOR THE TREATMENT OF PSYCHIATRIC AND NEUROLOGICAL DISORDERS

(76) Inventor: Effat Emamian, Short Hills, NJ (US)

(21) Appl. No.: 12/887,641

(22) Filed: Sep. 22, 2010

Related U.S. Application Data
(60) Provisional application No. 61/244,801, filed on Sep. 22, 2009.

(51) Int. Cl. G01N 33/00; A01K 67/00

(52) U.S. Cl. 800/3; 800/13; 800/18

(57) ABSTRACT

The present invention relates to mutant animals having a modified NMDA Receptor and use of the same for screening compounds useful for treating psychiatric and neurological disorders, such as schizophrenia, autism and Alzheimer's Disease. In particular, the invention provides an animal model that has mutation in phosphorylation of the NR1 subunit of the NMDAR, which causes behavioral deficits associated with psychiatric disorders useful for the evaluation of the therapeutic effects of psychotropic drugs for the treatment of these disorders.

### CROSS-REFERENCE TO RELATED APPLICATIONS

The present application claims the benefit under 35 USC 119(e) of U.S. Provisional Patent Application Ser. No. 61/244,801, filed: Sep. 22, 2009, entitled: Compositions and Methods for the Treatment of Psychiatric and Neurological Disorders, which is incorporated herein by reference in its entirety for all purposes.

### FIELD OF THE INVENTION

The present invention relates to mutant animals having a modified NMDA Receptor and use of the same for screening compounds useful for treating psychiatric and neurological disorders, such as schizophrenia, autism and Alzheimer's Disease.

### INCORPORATION BY REFERENCE

A Computer Readable Form of the Sequence Listing is filed herewith: file name: ESE_{—}5_seqlist_ST25.txt; size 31 KB; created on: Sep. 21, 2010; using PatentIn-3.5, and Checker 4.4.0 is hereby incorporated by reference in its entirety.

### BACKGROUND

The NMDA-type of glutamate receptors play an essential role in the induction of synaptic plasticity (R. Malinow and R. C. Malenka, 2002), which is believed to be the cellular mechanism underlying many forms of adaptive behaviors (H. W. Kessels and R. Malinow, 2009). Malfunctioning of NMDARs, on the other hand, has been implicated in major psychiatric and neurological disorders, such as schizophrenia and Alzheimer's disease (C. G. Lau and R. S. Zukin, 2007).

A prominent hypothesis of schizophrenia invokes hypofunction of the NMDAR (J. T. Coyle et al., 2003; J. T. Coyle and G. Tsai, 2004). Several lines of evidence supports this hypothesis. First, administration of non-competitive NMDAR antagonists, such as PCP or ketamine to healthy individuals produces the positive, negative, and cognitive symptoms that mimic schizophrenia, and induces and exacerbates those symptoms in schizophrenia patients (D. C. Javitt and S. R. Zukin, 1991; J. H. Krystal et al., 1994; A. K. Malhotra et al., 1997; C. M. Adler et al., 1999; G. K. Thaker and W. T. Carpenter, Jr., 2001; M. Pietraszek, 2003). Second, results from in vivo brain imaging studies suggest that NMDAR function is decreased in the brains of schizophrenia patients (R. A. Bressan and L. S. Pilowsky, 2000; M. J. Millan, 2005; C. Abbott and J. Bustillo, 2006; L. S. Pilowsky et al., 2006); but see (R. A. Bressan et al., 2005). Third, several studies suggest that enhancing NMDAR function can alleviate schizophrenic symptoms (T. Matsui et al., 1995; G. Tsai et al., 1998; D. C. Javitt, 2004; M. J. Millan, 2005; J. T. Coyle, 2006) but see (H. J. Tuominen et al., 2005). Lastly, genetic studies have identified several schizophrenia-linked genes that are either directly or indirectly involved in controlling NMDAR function (P. J. Harrison et al., 2003; P. J. Harrison and D. R. Weinberger, 2005; C. A. Ross et al., 2006). Despite the progress in the field, there has been inconsistency regarding the nature of NMDAR changes that occur in schizophrenia (S. Grimwood et al., 1999; S. Nudmamud and G. P. Reynolds, 2001; C. Konradi and S. Heckers, 2003; S. L. Eastwood, 2004; M. Beneyto and J. H. Meador-Woodruff, 2008). The exact role of NMDAR dysfunction in the etiology of schizophrenia is also unclear.

The NMDAR is phosphorylated in the cytoplasmic tail of each of its subunits, including NR1 and NR2, and phosphorylation of NMDAR has emerged as an important mechanism regulating its trafficking and function (B. S. Chen and K. W. Roche, 2007). The NR1 subunit of NMDARs is phosphorylated at serine 897 by PKA (W. G. Tingley et al., 1997). In the frontal cortex and hippocampus of schizophrenia patients, the phosphorylation level of NR1 at S897 is markedly reduced (E. S. Emamian et al., 2004a). The functional significance of NR1 S897 phosphorylation in vivo remains elusive.

Whether changes in NR1 phosphorylation play a role in the pathogenesis of schizophrenia or that the decreased phosphorylation itself is a compensatory response to the chronic disease is unknown. Accordingly, there exists an ongoing need for the development of models of neurophysiology for the identification of modulators and treatments for psychological and cognitive disorders.

### SUMMARY

The invention relates to the surprising and unexpected discovery of genes, proteins, and processes involved in synaptic plasticity. Therefore, the present invention provides nucleic acids; polypeptides and bioactive portions thereof; nucleic acids complementary to nucleic acids provided by the invention; vectors and/or host cells comprising the same; fusion proteins; antibodies or antigen-binding fragments thereof. In particular the invention provides novel mutant organisms, e.g., rats and/or mice, in which the genetic sequence and/or expression of a gene is altered or modulated.

In certain embodiments the transgenic animal provided by the invention comprises a sequence alteration in a gene encoding a subunit of the NMDA receptor (NMDAR). In another embodiment, the gene is the NR1 subunit of NMDAR. In another embodiment, the sequence alteration changes the Serine residue at position 897, 896 or 890 of the NR1 protein to another amino acid. In certain embodiments the Serine residue at position 897, 896 or 890 of the NR1 protein is changed to Alanine. In certain embodiments the Serine residue at position 897, 896 or 890 of the NR1 protein is changed to Glutamate or Aspartate.

In another aspect, the present invention provides methods for the use of novel mutant animals comprising at least one genetic modification (e.g., at least one nucleotide substitution or deletion; a transgene encoding a protein or portion or domain thereof, a mutant or derivative thereof) that affects the NMDAR protein function or expression, which is useful in screening for agents to treat cognitive and psychiatric disorders. In additional aspects, the invention provides diagnostic assays and methods of screening for chemical compounds that modulate NMDAR function and/or expression; and/or NR1 or NR2 function and/or expression. Therefore, the present invention provides compositions and methods useful for the identification of modulators and treatments for psychological and cognitive disorders. In particular, several psychiatric and neurological disorders that are manifested by cognitive dysfunction, including but not limited to schizophrenia, autism, Alzheimer's Disease and different type of dementias.

In another aspect, the invention provides compounds that can modulate the activity, transcription and/or translation (i.e., expression) of NMDAR or one of its subunits. As such, the targeting and modulating of NMDAR activity, and/or NR1 and/or NR2 gene (SEQ ID NOs: 2 and 4) expression, polypeptide synthesis, activity or protein-protein interactions represents a novel therapeutic intervention for treating pathologies relating to neurological dysfunction, including, for example, cognitive disorders and behavioral disorders.

The present invention further provides any invention described herein.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference, and for convenience are listed in the appended bibliography.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention.

FIG. 1. Genetic Blockade of NR1 S897 Phosphorylation. (A) Schematic view of the targeting vector generated for targeting the S897 site of GRIN1 (SEQ ID NOs: 1 and 2) locus of the mouse genome (see methods section). (B) Confirmation of gene targeting in the genomic DNA of the mutant mice by sequencing using a specific primer close to this site. (C) Western blot analysis to confirm the absence of phosphorylation at S897 in the S897A mutant animals using the NR1 S897 phospho-specific antibody. Total protein extracts of 50 •g from the frontal cortex (FC), striatum (STR) and hippocampus (HIP) of the wild type animals (WT) and homozygous mutants (HOM) were loaded. Upper blot: anti NR1 S897; middle blot: anti NR1; bottom blot: anti actin.

FIG. 2. The NR1 S897A Mutation Depressed Synaptic Transmission and Reduced LTP. (A) Upper panel shows the representative traces of evoked EPSCs recorded from CA1 neurons in either wild-type (WT) or mutant hippocampal slices. EPSCs at both −60 and +40 mV holding potentials are shown. Scale bars: 50 ms and 20 pA. Lower histogram is the quantification of the ratio of NMDAR to AMPAR-mediated synaptic transmission for WT and mutant animals (WT: 0.82±0.14, n=10; mutant mice: 0.31±0.04, n=12; **p<0.01, t-test). (B) Representative traces of AMPAR-mediated mEPSCs recorded from slices of WT (left) or mutant (right) animals (scale bars: 500 ms and 20 pA). Quantification of the amplitude of AMPAR-mediated mEPSCs for WT and mutant animals is described here (WT: 13.5±0.2 pA, n=1138 events from 10 cells; mutant mice: 12.4±0.2 pA, n=662 events from 10 cells; **p<0.01, K-S test). Quantification of the frequency of AMPAR mediated mEPSCs for WT and mutant animals is described here. (WT: 13.8±2.1 per min, n=10 cells; mutant: 8.5±1.4 per min, n=10 cells; *p<0.05, t-test). (C) Cumulative distribution of the inter-event intervals of the mEPSCs from WT and mutant animals (WT: n=1138 events from 10 cells; mutant mice: n=662 events from 10 cells; p<0.01; K-S test). (D) Upper panel: representative traces of field EPSPs (fEPSP) from WT or mutant hippocampal slices. Traces are averaged for time points before (1) and after (2) LTP induction. Scale bars: 200 ms and 0.1 mV. Lower panel: normalized amplitudes of fEPSPs before and after delivery of the LTP-induction stimuli (arrow). N=13 for both WT and mutant animals, *P<0.05.

FIG. 3. The NR1 S897A Mutation Decreased Synaptic Incorporation of Glutamate Receptors and Reduced GluR1 in the Synapse. (A) Left: Western blot analysis after biochemical fractionation of hippocampal tissues dissected from WT and the homozygous mutant mice. Synaptic membrane associated proteins (10 •g) were loaded and the same blot was probed with antibodies against GluR1 (top), NR1 (middle), and PSD-95 (bottom). Right: quantification of the densities of GluR1 (top) and NR1 (bottom) signals from WT (n=3) and the homozygous mutant (n=3) mice. (B) Immuno-EM analysis of the CA1 regions of the hippocampus from WT and mutant mice. Left panel: no primary antibody controls; Right panels, upper: three representative EM fields of the CA1 regions of wild-type animals that were probed with an anti-GluR1 antibody. The dark black staining of GluR1 in postsynaptic areas is the specific GluR1 signal, which is absent in the sections that were probed with the secondary antibody alone (no primary antibody control; on the left). Right panels, lower: three representative EM fields of the CA1 regions of the homozygous mutant mice probed with the anti-GluR1 antibody. The dark black staining shows the mislocalized, clusters of GluR1 signal. This signal is specific as it is absent in control sections (probed with the secondary antibody alone; on the left). Quantification of the number of GluR1 positive synapses in each EM field (2 •²) in the CA1 regions of the wild-type mice and homozygous mutant mice shows a highly significant decrease in the number of GluR1 positive synapses in mutants (***p<0.0001, t-test, N=9 for both groups). Scale bars: 200 nm.

FIG. 4. The NR1 S897A Mutation Causes Behavioral Deficits. (A&B) Social interaction of the experimental mice toward the repetitively presented stimulus mouse (test 1-4), or a novel stimulus mouse (test 5). Mutant: NR1 S897A homozygous mutant mice; WT: wild-type littermates. (A) Quantification of active social investigation: number of sniffs; N=8 for both groups. One Way Repeated Measures ANOVA (RMANOVA) was employed to evaluate recognition memory in WT and mutant mice. Significance (p<0.0001, F=8.3) was further evaluated using Bonferroni's multiple comparison post hoc test. In test 5, where a new intruder was introduced, WT mice showed increased recognition memory compared to mutant mice (p<0.001, t=5.728). WT mice also showed statistically decreased exploration toward the same intruder through tests 1-4 (p<0.01, t=4.226, test 1 vs. test 2; p<0.001, t=5.449, test 1 vs. test 3 and p<0.001, t=5.708 test 1 vs. test 4), whereas there was no significant difference when compared test 1 (the very first presentation of the intruder, which was presented repeatedly in tests 1-4) with test 5 (the new intruder). In contrary to WT mice, mutant mice did not show recognition memory throughout tests 1-5. (B) Quantification of the activity in exploring the cage away from social interest: number of rears (not in relation to the cylinder); N=8 for both groups. Compared to mutant mice, WT mice littermate exhibited significantly higher exploratory activity in test 1 (p<0.01, t=3.4). (C) Prepulse inhibition of NR1 S897A phosphomutant mice and WT littermate controls. PPI was expressed as 100-[(response to startle stimulus following prepulse/response to startle stimulus alone)×100]. *p<0.05, N=10 for both groups.

### DETAILED DESCRIPTION

The present invention is based upon the surprising and unexpected discovery that modulating the phosphorylation of the cytoplasmic tail of an NMDAR subunit (i.e., NR1 (SEQ ID NO: 1) and/or NR2 (SEQ ID NO: 3)) affects glutamatergic function. In particular, mutant animals that contain a mutation that alters the phosphorylation state of the NR1 subunit of the NMDAR demonstrate alterations in synaptic plasticity, cognitive and behavioral characteristics, e.g., those associated with psychiatric disorders.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). As used herein, the following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

The term “about” as it is used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

As used herein, “derivatives” are compositions formed from the native compounds either directly, by modification, or by partial substitution. As used herein, “analogs” are compositions that have a structure similar to, but not identical to, the native compound.

The term “NMDAR” is used in a general sense to refer to NMDAR polynucleotides or polypeptides, respectively, and unless indicated otherwise, encompasses the NR1 and NR2 subunits (See SEQ ID NOs.: 1 and 2), biologically-active fragments, portions, splice variants, and homologs thereof.

The term “NMDAR antagonist” or “antagonist of NMDAR” is used generally to refer to an agent capable of direct or indirect inhibition of NMDAR protein function, gene transcription, and/or translation (i.e., expression). The term “NMDAR agonist” or “agonist of NMDAR” is used generally to refer to an agent capable of direct or indirectly increasing NMDAR protein function, gene transcription, and/or translation.

The term “polypeptides” can mean, but is in no way limited to, recombinant full length, pro- and/or mature polypeptide forms as well as the biologically active forms, including fragments or splice variants, or recombinantly made truncations or portions derived from the full length polypeptides. Furthermore, polypeptides of the invention may include amino acid mimentics, and analogs. Recombinant forms of the chimeric polypeptides can be produced according to standard methods and protocols which are well known to those of skill in the art, including for example, expression of recombinant proteins in prokaryotic and/or eukaryotic cells followed by one or more isolation and purification steps, and/or chemically synthesizing cytokine polypeptides or portions thereof using a peptide synthesizer.

The term, “biologically active” or “bioactive” can mean, but is in no way limited to, the ability of an agent, such as the polypeptides provided by the invention, to effectuate a physiological change or response. The response may be detected, for example, at the cellular level, for example, as a change in gene expression, protein quantity, protein modification, protein activity, or combination thereof; at the tissue level; at the systemic level; or at the organism level. Techniques used to monitor these phenotypic changes include, for example, measuring: the binding of a ligand to its receptor in or on a cell, activation of cell signaling pathways, stimulation or activation of a cellular response, secretion or release of bioactive molecules from the cell, cellular proliferation and/or differentiation, animal behavior or a combination thereof.

The term “fragment” can mean, but is in no way limited to, sequences of at least 6 (contiguous) nucleic acids or at least 4 (contiguous) amino acids, a length sufficient to allow for specific hybridization in the case of nucleic acids or for specific recognition of an epitope or retention of a desired bioactivity in the case of amino acids, and are at most some portion less than a full length sequence.

The term “effective amount/dose,” “pharmaceutically effective amount/dose,” “pharmaceutically effective amount/dose” or “therapeutically effective amount/dose” can mean, but is in no way limited to, that amount/dose of the active pharmaceutical ingredient sufficient to prevent, inhibit the occurrence, ameliorate, delay or treat (alleviate a symptom to some extent, preferably all) the symptoms of a condition, disorder or disease state. The effective amount depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the agent. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the agents provided by the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

The term “nucleotide” can mean, but is no way limited to, a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

The term “nucleic acid” or “polynucleotide” can mean, but is in no way limited to, a molecule having more than one nucleotide, and is intended to include DNA molecules (e.g., cDNA or genomic DNA), RNA molecules, analogs of DNA or RNA, including locked nucleic acids and peptide nucleic acids, and derivatives thereof. The nucleic acid can be single, double, or multiple stranded and can comprise modified or unmodified nucleotides or non-nucleotides or various mixtures and combinations thereof. The nucleic acids of the invention are added directly, or can be complexed with cationic lipids, packaged within liposomes, or otherwise delivered to target cells or tissues. The nucleic acid or nucleic acid complexes can be locally administered to relevant tissues in vitro, ex vivo, or in vivo through injection or infusion pump, with or without their incorporation in biopolymers.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

The term “modified bases” can mean, but is in no way limited to, nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule. The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163).

The term “hybridization” can mean, but is in no way limited to, the binding, duplexing, or hybridizing of a molecule only to a particular nucleotide sequence under low, medium, or highly stringent conditions, including when that sequence is present in a complex mixture (e.g., total cellular) DNA or RNA.

The term “conservative mutations” refers to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous. In certain embodiments, the invention relates to functional mutations in which an amino acid has been changed or modified to deleted, add or mimic a post-translational modification, e.g., phosphorylation. For example, a Serine/Threonine residue that is normally phosphorylated can be mutated to, e.g., an Alanine to mimic the unphosphorylated state; or mutated to an acidic residue to mimic the phosphorylated state.

The term “down-regulate” can mean, but is in no way limited to, the expression of the gene, or level of RNAs or equivalent RNAs encoding one or more proteins, or activity of one or more proteins is reduced below that observed in the absence of an agent provided by the invention. For example, the expression of a gene can be decreased in order to treat, prevent, ameliorate, or modulate a pathological condition caused or exacerbated by high levels of gene expression.

The term “up-regulate” can mean, but is in no way limited to, the expression of the gene, or level of RNAs or equivalent RNAs encoding one or more protein subunits, or activity of one or more protein subunits is greater than that observed in the absence of an agent provided by the invention. For example, the expression of a gene can be increased in order to treat, prevent, ameliorate, or modulate a pathological condition caused or exacerbated by an absence or low level of gene expression.

The term, “modulate” can mean, but is in no way limited to, the function or expression of the gene or level of RNAs or equivalent RNAs encoding one or more proteins is altered, e.g., reduced or increased.

The term, “gene” can mean, but is in no way limited to, a nucleic acid that encodes RNA, for example, nucleic acid sequences including but not limited to a segment encoding a polypeptide.

The term “complementarity” can mean, but is in no way limited to, the ability of a nucleic acid to form hydrogen bond(s) with another RNA sequence by either traditional Watson-Crick, Hoogsteen base pairing or other non-traditional types.

The term “binding” can mean, but is in no way limited to, the physical or chemical interaction, direct or indirect, between two molecules (e.g., compounds, amino acids, nucleotides, polypeptides, or nucleic acids). Binding includes covalent, hydrogen bond, ionic, non-ionic, van der Waals, hydrophobic interactions, and the like.

The NMDAR is a specific type of ionotropic glutamate receptor. NMDA (N-methyl D-aspartate) is the name of a selective agonist that binds to NMDA receptors but not to other glutamate receptors. The NMDA receptor forms a heterotetramer between two NR1 (SEQ ID NO: 1) and two NR2 (SEQ ID NO:3) subunits; two obligatory NR1 subunits and two regionally localized NR2 subunits. A related gene family of NR3 A and B subunits have an inhibitory effect on receptor activity. Multiple receptor isoforms with distinct brain distributions and functional properties arise by selective splicing of the NR1 transcripts and differential expression of the NR2 subunits.

Each receptor subunit has modular design and each structural module also represents a functional unit: the extracellular domain contains two globular structures: a modulatory domain and a ligand-binding domain. NR1 subunits bind the co-agonist glycine and NR2 subunits bind the neurotransmitter glutamate. The agonist-binding module links to a membrane domain, which consists of three trans-membrane segments and a re-entrant loop reminiscent of the selectivity filter of potassium channels. The membrane domain contributes residues to the channel pore and is responsible for the receptor's high-unitary conductance, high-calcium permeability, and voltage-dependent magnesium block. Each subunit has an extensive cytoplasmic domain, which contain residues that can be directly modified by a series of protein kinases and protein phosphatases, as well as residues that interact with a large number of structural, adaptor, and scaffolding proteins.

The NMDA receptor (NMDAR), a glutamate receptor, is the predominant molecular device for controlling synaptic plasticity and memory function. Calcium flux through NMDARs is thought to play a critical role in synaptic plasticity, a cellular mechanism for learning and memory. The NMDA receptor is distinct in two ways: first, it is both ligand-gated and voltage-dependent; second, it requires co-activation by two ligands-glutamate and glycine. NMDAR is phosphorylated in the cytoplasmic tail of each of its subunits, including NR1 and NR2. Phosphorylation of the NR1 subunit of NMDA receptors (NMDAR) is markedly reduced in schizophrenia patients. However, the role of NR1 phosphorylation at, e.g., S890, S896, and/or S897 (see SEQ ID NO: 1) in normal synaptic function and adaptive behaviors was, heretofore, unknown.

Activation of NMDA receptors results in the opening of an ion channel that is nonselective to cations. A unique property of the NMDA receptor is its voltage-dependent activation, a result of ion channel block by extracellular Mg²⁺ ions. This allows voltage-dependent flow of Na⁺ and small amounts of Ca²⁺ ions into the cell and K⁺ out of the cell. Activation of NMDA receptors requires binding of glutamate or aspartate (aspartate does not stimulate the receptors as strongly). In addition, NMDARs also require the binding of the co-agonist glycine for the efficient opening of the ion channel, which is a part of this receptor.

D-serine has also been found to co-agonize the NMDA receptor with even greater potency than glycine. D-serine is produced by serine racemase, and is enriched in the same areas as NMDA receptors. Removal of D-serine can block NMDA-mediated excitatory neurotransmission in many areas. Recently, it has been shown that D-serine is synthesized mostly by glial cells, indicating a role for glia-derived D-serine in NMDA receptor regulation.

In addition, a third requirement is membrane depolarization. A positive change in transmembrane potential will make it more likely that the ion channel in the NMDA receptor will open by expelling the Mg²⁺ ion that blocks the channel from the outside. This property is fundamental to the role of the NMDA receptor in memory and learning, and it has been suggested that this channel is a biochemical substrate of Hebbian learning, where it can act as a coincidence detector for membrane depolarization and synaptic transmission.

Antagonists of the NMDA receptor are used as anesthetics for animals and sometimes humans, and are often used as recreational drugs due to their hallucinogenic properties, in addition to their unique effects at elevated dosages such as dissociation. When NMDA receptor antagonists are given to rodents in large doses, they can cause a form of brain damage called Olney's Lesions. So far, the published research on Olney's Lesions is inconclusive in its occurrence upon human or monkey brain tissues with respect to an increase in the presence of NMDA receptor antagonists.

NMDA receptor function is also strongly regulated by chemical reduction and oxidation, via the so-called “redox modulatory site.” Through this site, reductants dramatically enhance NMDA channel activity, whereas oxidants either reverse the effects of reductants or depress native responses. It is generally believed that NMDA receptors are modulated by endogenous redox agents such as glutathione, lipoic acid, and the essential nutrient pyrroloquinoline quinone. Src kinase enhances NMDA receptor currents. http://en.wikipedia.org/wiki/NiMDAR-cite_note-pmid9005855-20#cite_note-pmid9005855-20 Reelin modulates NMDA function through Src family kinases and DAB1 significantly enhancing LTP in the hippocampus; CDK5 regulates the amount of NR2B-containing NMDA receptors on the synaptic membrane, thus affecting synaptic plasticity.

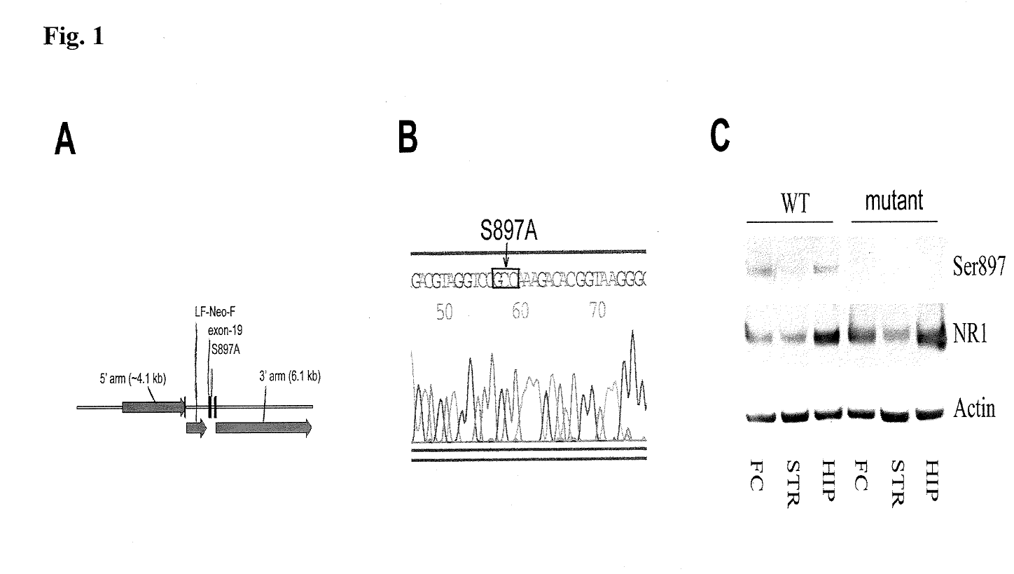

Presently, the physiological and behavioral role of NR15897 phosphorylation in vivo was examined in order to gain insight into the link between the decreased phosphorylation at this site and abnormal behaviors. To address these questions mice were generated in which the NR1 S897 is replaced with alanine (A) (FIG. 1A, see methods section), thereby preventing its phosphorylation. We confirmed that the mutant mice carry the point mutation in its genome (FIG. 1B), express the full-length NR1 protein at a level comparable to the wild type mice (FIG. 1C), and the phosphorylation of NR1 at S897 is precluded due to the mutation to alanine (FIG. 1C).

This knock-in mutation causes severe impairment in NMDAR synaptic incorporation and NMDAR-mediated synaptic transmission. Furthermore, the phosphomutant animals have reduced AMPA receptor (AMPAR)-mediated synaptic transmission, decreased AMPAR GluR1 subunit in the synapse, and impaired long-term potentiation (LTP). Finally, the mutant mice exhibit behavioral deficits in social interaction and sensorimotor gating. The results suggest that an impairment in NR1 phosphorylation leads to glutamatergic hypofunction that can contribute to behavioral deficits associated with psychiatric disorders.

Nucleic Acids

The various aspects and embodiments described below include nucleic acids encoding NMDAR polypeptides (e.g., NR1 and/or NR2; SEQ ID NOs: 1 and 2) and/or bioactive portions and fragments thereof, as well as genes which encode NMDAR polypeptides, including homologs, orthologs, and paralogs, isoforms, splice variants, and polymorphisms. Those additional genes can be analyzed for target sites using the methods described herein. Thus, the inhibition and the effects of such inhibition of the other genes can be performed as described herein.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47.

Unless otherwise indicated, nucleic acid compositions provided by the invention, including NR1 nucleic acids and/or NR2 nucleic acids, are collectively and interchangeably referred to herein as “NMDAR nucleic acids” or “NMDAR polynucleotides”, and the corresponding encoded polypeptides are referred to as “NMDAR polypeptides” or “NMDAR proteins.” Unless indicated otherwise, these terms include bioactive portions, fragments, deletions or substitutions, truncations, gene fusions at the amino or carboxy terminal or both, and combinations thereof.

In another aspect, the invention provides derivatives and/or analogs of the NMDAR nucleic acids as set forth in SEQ ID NOs: 2 and 4, and/or the NMDAR proteins as set forth in SEQ ID NOs: 1 and 3 of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

In any of the embodiments described herein, the nucleic acids encoding an NMDAR can be present as: one or more naked DNAs; one or more nucleic acids disposed in an appropriate expression vector and maintained episomally; one or more nucleic acids incorporated into the host cell's genome; a modified version of an endogenous gene encoding the components of the complex; one or more nucleic acids in combination with one or more regulatory nucleic acid sequences; or combinations thereof. The nucleic acid may optionally comprise a linker peptide or fusion protein component, for example, His-Tag, FLAG-Tag, Maltose Binding Protein (MBP)-Tag, fluorescent protein, GST, TAT, an antibody portion, a signal peptide, and the like, at the 5′ end, the 3′ end, or at any location within the ORF.

In an additional aspect, the invention provides antisense and/or interfering nucleic acids (e.g., RNAi) capable of specifically targeting NMDAR nucleic acids (e.g., SEQ ID NOs: 2 or 4). For example, the present invention features a nucleic acid molecule, such as a decoy RNA, dsRNA, siRNA, shRNA, microRNA, aptamer, and/or antisense nucleic acid molecules, which down regulates expression of a sequence encoding an NMDAR protein. In another embodiment, a nucleic acid molecule of the invention has an endonuclease activity or is a component of a nuclease complex, and cleaves RNA having an NMDAR nucleic acid sequence.

In any of the interfering nucleic acid embodiments, the nucleic acid molecule comprises between 12 and 100 bases complementary to an RNA having an NMDAR nucleic acid sequence. In another embodiment, the nucleic acid molecule comprises between 14 and 24 bases complementary to an RNA having an NMDAR nucleic acid sequence. In any embodiment described herein, the nucleic acid molecule can be synthesized chemically according to methods well known in the art. A number of references describe useful methods and approaches for generating RNAs including: U.S. Pat. Nos. 6,900,187, 6,383,808, 7,101,991, 7,285,541, 7,368,436, 7,022,828; which are incorporated herein by reference.

In another embodiment, the inhibitory RNA is at least one of an antisense RNA, an interfering RNA or a combination of both. In yet another embodiment, the interfering RNA is at least one of a siRNA, a miRNA or a combination of both. In another aspect the invention provides a nucleic acid vector comprising any of the inhibitory nucleic acids described herein. In additional aspects, the invention provides a host cell comprising any of the inhibitory nucleic acids described herein and/or any of the vectors provided by the invention.

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs and/or microRNAs (miRNA).

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

Mutant Animals

As used herein, the term “mutant animals” is used broadly and includes animals that have been engineered to carry one or more transgenes (i.e., transgenic), and mutant animals whose genome has been modified such that an endogenous genetic sequence has been altered (e.g., substitution, mutation, deletion, or the like), including knock-ins, knock-outs or knock-downs, and animals whose genome has been modified such that an endogenous gene is upregulated or overexpressed.

One particularly useful application of the invention is the generation of novel mutant animals, such as mice, to model different neurological diseases, e.g., neurodegenerative diseases, cognitive disorders, and psychiatric disorders, in particular, schizophrenia. Such mutant mice will have utility in developing specific and general therapies and screening methods to identify novel compounds and to otherwise employ the general inventive aspects of the present invention.

Therefore, in an embodiment, the invention provides a mutant animal comprising a modified NMDA Receptor, wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1. In another embodiment, the animal is a mouse. In certain embodiments the amino acid substitution is at Serine 897. In still other embodiments, Serine 897 is substituted with Alanine. In further embodiments, Serine 897 is substituted with Glutamate or Aspartate.

Mutant mice can be produced by techniques known in the art, e.g., microinjection, as described in, e.g., U.S. Pat. No. 4,736,866 issued to Leder et al., and/or as provided by B. Hogan et al. entitled “Manipulating the Mouse Embryo: A Laboratory Manual”, Cold Spring Harbor Laboratory, Cold Spring Harbor, N.Y., U.S.A. (1986). U.S. Pat. No. 5,574,206 issued to Jolicoeur particularly describes the creation of mutant mice bearing functional HIV genes and their use in the modeling and study of HIV-mediated diseases. These references are herein incorporated by reference.

In certain aspects, the invention provides a non-human mutant animal containing an alteration or mutation in at least one gene of interest (GOI). In certain embodiments, the gene is an endogenous gene, for example, the NR1 or NR2 gene of the NMDAR. In other embodiments, the GOI is no longer produced, is overexpressed, or is altered and/or modified such that one or more amino acids are different from the wild type (WT) version of the gene. In an additional embodiment, the animal is capable of expressing the altered gene. In certain additional embodiments, the mutant animal contains an exogenous nucleic acid, e.g., a promoter sequence or a gene, which may also be incorporated into the animals genome. In another embodiment, the altered gene is under the control of an inducible promoter.

In another embodiment, the invention provides a non-human mutant animal comprising at least one alteration in the NR1 gene such that the animal expresses a NR1 protein in which phosphorylation of 5897 is reduced or eliminated. In certain embodiments, the alteration results in an Alanine residue at position 897 (i.e., S897A). In another preferred embodiment, the invention provides a non-human mutant animal comprising at least one alteration in the NR1 gene such that the animal expresses a NR1 protein that has an acidic amino acid residue at position 897; i.e., a mutation that mimics constitutive phosphorylation of S897 (i.e., S897 Glu or 5987 Asp).

In another embodiment, embryonic stem cells can be derived from the non human mutant animal of the foregoing embodiments. In another embodiment progeny can also be derived from the non human mutant animal of any of the foregoing embodiments. In preferred embodiment, the invention provides a method of making a non-human mutant animal from embryonic stem cells wherein the endogenous NR1 gene is altered such that the product of the NR1 gene comprises a S897A mutation. In certain embodiments, the non-human mutant animal in any the foregoing embodiments is a mouse, rat, rabbit or goat. The most preferred animal of the foregoing group is a mouse.

Alteration or modification of an endogenous GOI in a cell can be achieved by homologous recombination between the allele and a GOI gene, or portion thereof, introduced into the cell. The cell can be a cell type that normally expresses the GOI. Alternatively, the cell can be a pluripotent progenitor cell that can develop into an animal, such as an embryonic stem cell. When the cell is an embryonic stem cell, the cell can be introduced into a blastocyst, and the blastocyst allowed to develop in a foster animal to thereby produce an animal having somatic and germ cells in which an endogenous GOI gene allele is functionally modified. Such an animal is referred to herein as a “homologous recombinant” animal. A preferred homologous recombinant animal of the invention is a mouse.

To create a homologous recombinant cell or animal, a targeting vector is prepared which contains DNA encoding a GOI or a portion thereof, having a mutation introduced therein. A preferred targeting vector for creating a null mutation in an endogenous GOI includes GOI-encoding DNA into which has been inserted non-GOI encoding DNA. Thus, the non-homologous replacement portion is flanked 5′ and 3′ by nucleotide sequences with substantial identity to the GOI. A nucleotide sequence with “substantial identity” to a GOI sequence is intended to describe a nucleotide sequence having sufficient homology to a GOI sequence to allow for homologous recombination between the nucleotide sequence and an endogenous GOI sequence in a host cell. Typically, the nucleotide sequences of the flanking homology regions are at least 80%, more preferably at least 90%, even more preferably at least 95%, and most preferably 100% identical to the nucleotide sequences of the endogenous GOI to be targeted for homologous recombination. Most preferably, the flanking homology regions are isogenic with the targeted endogenous allele (e.g., the DNA of the flanking regions is isolated from cells of the same genetic background as the cell into which the targeting construct is to be introduced). Additionally, the flanking homology regions of the targeting vector are of sufficient length for homologous recombination between the targeting vector and an endogenous GOI gene in a host cell when the vector is introduced into the host cell. Typically, the flanking homology regions are at least 1 kilobase in length and more preferably are least several kilobases in length.

A typical targeting vector has a positive selection expression cassette as the non-homologous replacement portion. The term “positive selection expression cassette” refers to nucleotide sequences encoding a positive selection marker operatively linked to regulatory elements that control expression of the positive selection marker (e.g., promoter and polyadenylation sequences). A “positive selection marker” allows for selection of cells which contain the marker, whereas cells that do not contain and express the marker are selected against (e.g., are killed by the selecting agent). For example, a preferred positive selection expression cassette includes a neomycin phosphotransferase (“neo”) gene operatively linked to a promoter and a polyadenylation signal. Cells carrying and expressing the neo gene exhibit resistance to the selecting agent G418.

In addition to the positive selection expression cassette, a targeting vector of the invention typically also includes a negative selection expression cassette located distal to either the upstream or downstream homology regions (i.e., the regions substantially identical to Ig-encoding sequences). A “negative selection expression cassette” refers to nucleotide sequences encoding a negative selection marker operatively linked to regulatory elements that control expression of the negative selection marker. A “negative selection marker” allows for selection against cells which carry the marker, e.g., cells that contain and express the marker are killed by a selecting agent, whereas cells that do not contain and express the negative selection marker survive. For example, a negative selection expression cassette includes a herpes simplex virus thymidine kinase (“tk”) gene operatively linked to a promoter and a polyadenylation signal. Cells that contain and express the tk gene can be killed, for example, by the selecting agent gancyclovir.

This configuration of the targeting vector allows for use of the “positive/negative” selection technique for selecting homologous recombinants: cells into which the targeting vector has been introduced are selected that contain and express the positive selection marker but which have lost the negative selection marker. Accordingly, these cells carry the non-homologous replacement portion DNA (e.g., the inserted neo gene) but have lost the DNA encoding the negative selection marker located distal thereto in the targeting vector, likely as a result of homologous recombination between the targeting vector and the endogenous gene.

In a preferred embodiment, the targeting vector includes flanking homology regions having substantial identity to a mouse GOI sequences to thereby target an endogenous mouse GOI in a mouse host cell (e.g., a murine embryonic stem cell) for homologous recombination. Murine GOI genomic DNA used as the flanking homology regions of the targeting vector can be isolated from a murine genomic DNA library by screening the library with a cDNA probe encompassing all or part of the murine GOI cDNA cDNA using standard techniques. Preferably, a genomic DNA library screened is prepared from cells isogenic with the cell to be transfected with the targeting vector. For example, a genomic library from the 129/Sv strain of mouse (available commercially from Stratagene) can be screened to isolate mouse Ig genomic DNA for use in a targeting vector for transfection into the D3 embryonic stem cell line derived from strain 129/Sv.

To functionally disrupt an endogenous GOI allele in a host cell, a targeting vector of the invention is introduced into the host cell, e.g., a differentiated cell that normally expresses the GOI, or an embryonic stem cell, and homologous recombinants are selected. A targeting vector can be introduced into a host cell by any of several techniques known in the art suitable for the introduction of exogenous DNA (e.g., calcium phosphate precipitation, DEAE-dextran transfection, microinjection, lipofection or electroporation, and the like. After introduction of the vector into the host cell, the cell is cultured for a period of time and under conditions sufficient to allow for homologous recombination between the introduced targeting vector and an endogenous GOI. Host cells are selected (e.g., by the positive/negative selection techniques described above) and screened for homologous recombination at the endogenous GOI locus by standard techniques (e.g., Southern hybridizations using a probe which distinguishes the normal endogenous allele from the homologous recombinant allele).

To create a homologous recombinant animal of the invention, an embryonic stem cell having one GOI allele functionally disrupted is introduced into a blastocyst, the blastocyst is implanted into a pseudopregnant foster mother, and the embryo allowed to develop to term. The resultant animal is a chimera having cells descendant from the embryonic stem cell. Chimeric animals in which the embryonic stem cell has contributed to the germ cells of the animal can be mated with wild type animals to thereby produce animals heterozygous for the GOI gene disruption in all somatic and germ cells. The heterozygous animals can then be mated to create animals homozygous for the gene disruption (i.e., having both GOI alleles functionally disrupted). These homologous recombinant animals mentioned above can be used as control or test animals for in vivo screening assays (described further in detail below). Additionally, cells of the animal homozygous for the GOI disruption can be isolated from the animals and cultured for use in in vitro screening assays. Furthermore, immortalized cell lines can be prepared from cells of the animal using standard techniques for cell immortalization, e.g., by transfection of the cells with an expression vector encoding myc, ras or SV40 large T antigen.

For additional descriptions of targeting vectors and homologous recombination methodologies, see also e.g., Thomas, K. R. et al. (1986) Cell 44:419-428; Thomas, K. R. et al. (1987) Cell 51:503-512; Thomas, K. R. et al. (1992) Mol. Cell. Biol. 12:2919-2923; Deng, C. and Capecchi, M. R. (1992) Mol. Cell. Biol. 12:3365-3371; Hasty, P. et al. (1992) Mol. Cell. Biol. 12:2464-2474; Li, E. et al. (1992) Cell 69:915; Zhang, H., et al. (1994) Mol. Cell. Biol. 14:2404-2410; Bradley, A. in Teratocarcinomas and Embryonic Stem Cells: A Practical Approach, E. J. Robertson, ed. (IRL, Oxford, 1987) pp. 113-152; PCT International Publication No. WO 90/11354; PCT International Publication No. WO 91/01140; PCT International Publication No. WO 91/19796; PCT International Publication No. WO 92/20808; and PCT International Publication No. WO 93/04169. Alternatively, nuclei from somatic cells which are heterozygous or homozygous for the GOI disruption can be introduced into enucleated unfertilized eggs and subsequently implanted into pseudopregnant foster mothers to generate homologous recombinant animals, see also e.g., Wilmut, I. et al. (1997) Nature 385(6619):810-813; Kato, Y. et al. (1998) Science 282(5396):2095-2098; Wakayama, T. et al. (1998) Nature 394(6691):369-374; McCreath, K. J. et al. (2000) Nature 405(6790):1066-1069; Wakayama, T. et al (2001) Mol. Reprod. Dev. 58(4):376-383. Additionally, a recombinase can be used to functionally disrupt a GOI by homologous recombination as described in PCT International Publication WO 93/22443.

In addition to allowing for introduction of a null mutation in a gene allele, similar techniques can be used to introduce insertions, point mutations or deletions into a gene allele. Point or deletion mutations can be introduced into a gene allele by, for example, the “hit and run” homologous recombination procedure (as described in Valancius, V. and Smithies, O. (1991) Mol. Cell. Biol. 11:1402-1408; and Hasty, P. et al. (1991) Nature 350:243-246) or by the double replacement homologous recombination procedure (as described in Wu, H. et al. (1994) Proc. Natl. Acad. Sci. USA 91:2819-2823). Accordingly, in another embodiment, the invention provides homologous recombinant cells and animals (e.g., human cells or non-human animals) that express an altered GOI product.

To create a mutant animal, a nucleic acid of the invention encoding a transactivator fusion protein, as described above, can be incorporated into a recombinant expression vector in a form suitable for expression of the fusion protein in a host cell. The term “in a form suitable for expression of the fusion protein in a host cell” is intended to mean that the recombinant expression vector includes one or more regulatory sequences operatively linked to the nucleic acid encoding the fusion protein in a manner which allows for transcription of the nucleic acid into mRNA and translation of the mRNA into the fusion protein. The term “regulatory sequence” is art-recognized and intended to include promoters, enhancers and other expression control elements (e.g., polyadenylation signals). Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the amount of fusion protein to be expressed.

When used in mammalian cells, a recombinant expression vector's control functions are often provided by viral genetic material. For example, commonly used promoters are derived from polyoma, adenovirus 2, cytomegalovirus and simian virus 40. Use of viral regulatory elements to direct expression of the fusion protein can allow for high level constitutive expression of the fusion protein in a variety of host cells. In a preferred recombinant expression vector, the sequences encoding the fusion protein are flanked upstream (i.e., 5′) by the human cytomegalovirus IE promoter and downstream (i.e., 3′) by an SV40 poly(A) signal. For example, an expression vector similar to that described in Example 1 can be used. The human cytomegalovirus IE promoter is described in Boshart et al. (1985) Cell 41:521-530. Other ubiquitously expressing promoters which can be used include the HSV-Tk promoter (disclosed in McKnight et al. (1984) Cell 37:253-262) and •-actin promoters (e.g., the human •-actin promoter as described by Ng et al. (1985) Mol. Cell. Biol. 5:2720-2732).

Alternatively, the regulatory sequences of the recombinant expression vector can direct expression of the fusion protein preferentially in a particular cell type, i.e., tissue-specific regulatory elements can be used. Non-limiting examples of tissue-specific promoters which can be used include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the •-fetoprotein promoter (Campes and Tilghman (1989) Genes Dev. 3:537-546).

Alternatively, a self-regulating construct encoding a transactivator fusion protein can be created. To accomplish this, nucleic acid encoding the fusion protein is operatively linked to a minimal promoter sequence and at least one tet operator sequence. When this nucleic acid is introduced into a cell (e.g., in a recombinant expression vector), a small amount of basal transcription of the transactivator gene is likely to occur due to “leakiness”. In the presence of tetracycline Tc (or analog thereof) this small amount of the transactivator fusion protein will bind to the tet operator sequence(s) upstream of the nucleotide sequence encoding the transactivator and stimulate additional transcription of the nucleotide sequence encoding the transactivator, thereby leading to further production of the transactivator fusion protein in the cell. It will be appreciated by those skilled in the art that such a self-regulating promoter can also be used in conjunction with other tetracycline-regulated transactivators, such as the wild-type Tet repressor fusion protein (tTA) described in Gossen, M. and Bujard, H. (1992) Proc. Natl. Acad. Sci. USA 89:5547-5551, which binds to tet operators in the absence of Tc (as illustrated in FIG. 9A). When used in conjunction with this transactivator, self-regulated transcription of the nucleotide sequence encoding this transactivator is stimulated in the absence of Tc. The plasmid pUHD15-3, which comprises nucleotide sequences encoding the tTA described in Gossen and Bujard (1992), cited supra, operatively linked to a self-regulating promoter, has been deposited on Jul. 8, 1994 under the provisions of the Budapest Treaty at the Deutsche Sammlung Von Mikroorganismen and Zell Kulturen GmbH (DSM) in Braunschweig, Germany and assigned deposit number DSM 9280.

In one embodiment, the recombinant expression vector of the invention is a plasmid. Alternatively, a recombinant expression vector of the invention can be a virus, or portion thereof, which allows for expression of a nucleic acid introduced into the viral nucleic acid. For example, replication defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include •Crip, •Cre, •2 and •A m. The genome of adenovirus can be manipulated such that it encodes and expresses a transactivator fusion protein but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251-3260 can be used to express a transactivator fusion protein.

Nucleic acid encoding fusion proteins can be introduced into a host cell by standard techniques for transfecting eukaryotic cells. The term “transfecting” or “transfection” is intended to encompass all conventional techniques for introducing nucleic acid into host cells, including calcium phosphate co-precipitation, DEAE-dextran-mediated transfection, lipofection, electroporation and microinjection. Suitable methods for transfecting host cells can be found in Sambrook et al. (Molecular Cloning: A Laboratory Manual, 2nd Edition, Cold Spring Harbor Laboratory press (1989)), and other laboratory textbooks.

The number of host cells transformed with a nucleic acid of the invention will depend, at least in part, upon the type of recombinant expression vector used and the type of transfection technique used. Nucleic acid can be introduced into a host cell transiently, or more typically, for long term regulation of gene expression, the nucleic acid is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acid of interest. Preferred selectable markers include those which confer resistance to certain drugs, such as G418 and hygromycin. Selectable markers can be introduced on a separate plasmid from the nucleic acid of interest or, are introduced on the same plasmid. Host cells transfected with a nucleic acid of the invention (e.g., a recombinant expression vector) and a gene for a selectable marker can be identified by selecting for cells using the selectable marker. For example, if the selectable marker encodes a gene conferring neomycin resistance, host cells which have taken up nucleic acid can be selected with G418. Cells that have incorporated the selectable marker gene will survive, while the other cells die.

A host cell transfected with a nucleic acid encoding a fusion protein of the invention can be further transfected with one or more nucleic acids which serve as the target for the fusion protein. The target nucleic acid comprises a nucleotide sequence to be transcribed operatively linked to at least one tet operator sequence.

Nucleic acid encoding the fusion protein of the invention can be introduced into eukaryotic cells growing in culture in vitro by conventional transfection techniques (e.g., calcium phosphate precipitation, DEAE-dextran transfection, electroporation etc.). Nucleic acid can also be transferred into cells in vivo, for example by application of a delivery mechanism suitable for introduction of nucleic acid into cells in vivo, such as retroviral vectors (see e.g., Ferry, N et al. (1991) Proc. Natl. Acad. Sci. USA 88:8377-8381; and Kay, M. A. et al. (1992) Human Gene Therapy 3:641-647), adenoviral vectors (see e.g., Rosenfeld, M. A. (1992) Cell 68:143-155; and Herz, J. and Gerard, R. D. (1993) Proc. Natl. Acad. Sci. USA 90:2812-2816), receptor-mediated DNA uptake (see e.g., Wu, G. and Wu, C. H. (1988) J. Biol. Chem. 263:14621; Wilson et al. (1992) J. Biol. Chem. 267:963-967; and U.S. Pat. No. 5,166,320), direct injection of DNA (see e.g., Acsadi et al. (1991) Nature 332:815-818; and Wolff et al. (1990) Science 247:1465-1468) or particle bombardment (see e.g., Cheng, L. et al. (1993) Proc. Natl. Acad. Sci. USA 90:4455-4459; and Zelenin, A. V. et al. (1993) FEBS Letters 315:29-32). Thus, for gene therapy purposes, cells can be modified in vitro and administered to a subject or, alternatively, cells can be directly modified in vivo.

The nucleic acid transactivator fusion protein can be transferred into a fertilized oocyte of a non-human animal to create a mutant animal which expresses the fusion protein of the invention in one or more cell types. In one embodiment, the non-human animal is a mouse, although the invention is not limited thereto. In other embodiments, the mutant animal is a goat, sheep, pig, cow or other domestic farm animal. Such mutant animals are useful for large scale production of proteins (so called “gene pharming”). In still another embodiment, the mutant animal is a non-human primate.

A mutant animal can be created, for example, by introducing a nucleic acid encoding the fusion protein (typically linked to appropriate regulatory elements, such as a constitutive or tissue-specific enhancer) into the male pronuclei of a fertilized oocyte, e.g., by microinjection, and allowing the oocyte to develop in a pseudopregnant female foster animal. Intronic sequences and polyadenylation signals can also be included in the transgene to increase the efficiency of expression of the transgene. Methods for generating mutant animals, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory. A mutant founder animal can be used to breed additional animals carrying the transgene. Mutant animals carrying a transgene encoding the fusion protein of the invention can further be bred to other mutant animals carrying other transgenes, e.g., to a mutant animal which contains a gene operatively linked to a tet operator sequence.

It will be appreciated that, in addition to mutant animals, the regulatory system described herein can be applied to other mutant organisms, such as mutant plants. Mutant plants can be made by conventional techniques known in the art. Accordingly, the invention encompasses non-human mutant organisms, including animals and plants, that contains cells which express the transactivator fusion protein of the invention (i.e., a nucleic acid encoding the transactivator is incorporated into one or more chromosomes in cells of the mutant organism).

The features and characteristics of the animals of the invention, and cells derived therefrom, make them useful for a wide variety of applications, as described in further detail in the subsections below.

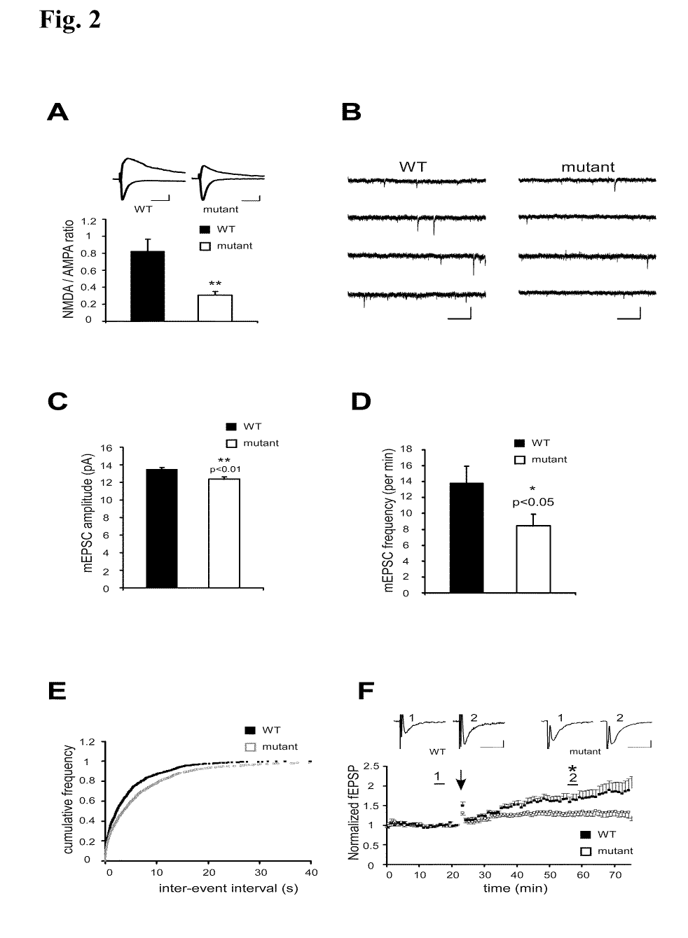

To examine the effects of NR1 S897 phosphomutation on synaptic function, we recorded both NMDAR and AMPAR-mediated synaptic transmission in the Schaffer collateral—CA1 synapses in the hippocampi of S897A mutant mice. Compared with wild-type animals, the ratio of NMDAR to AMPAR-mediated synaptic transmission was markedly reduced in the mutant animals (FIG. 2A; WT: 0.82±0.14, n=10; mutant mice: 0.31±0.04, n=12; p<0.01). The decrease in NMDA-to-AMPA ratio could be due to either a decrease of NMDAR, or an increase in AMPAR-mediated synaptic transmission. To distinguish between these possibilities, we recorded AMPAR-mediated miniature EPSCs (mEPSC) from CA1 pyramidal neurons in the hippocampus. We found that both the amplitude (FIG. 2 B; WT: 13.5±0.2 pA, n=1138 events from 10 cells; mutant mice: 12.4±0.2 pA, n=662 events from 10 cells; p<0.01 by K-S test), and the frequency (FIG. 2B; WT: 13.8±2.1 per min, n=10 cells; mutant: 8.5±1.4 per min, n=10 cells; p<0.05) of the AMPAR-mediated mEPSCs were decreased in the mutant animals. As expected, the inter-event intervals of mEPSCs from the mutant animals were longer (FIG. 2C; p<0.01 by K-S test). The reduction of mEPSC frequency likely reflects a decrease in the number of functional synapses that contain AMPARs, whereas the decrease in mEPSC amplitude is likely due to a reduction in the number of AMPARs per synapse. These results demonstrate that both NMDAR and AMPAR-mediated synaptic transmission onto CA1 cells are reduced. The reduction of NMDA-to-AMPA ratio indicates that the reduction of NMDA transmission is more dramatic than that of AMPA. As expected, given the important role of NMDARs in the induction of LTP, LTP in the Schaffer collateral—CA1 pathway was impaired in the mutant mice (FIG. 2D, n=13 for both WT and mutant animals, p<0.05). These results indicate a severe impairment of NMDAR-mediated synaptic transmission in the mutant animals, leading to decreased synaptic plasticity and AMPAR-mediated synaptic transmission. Since both AMPAR- and NMDAR-mediated synaptic transmission are markedly impaired, the deficiency of LTP in the mutant animals could be because there is insufficient activation of NMDARs (due to the impairment in AMPAR-mediated synaptic transmission) or insufficient number of NMDARs.

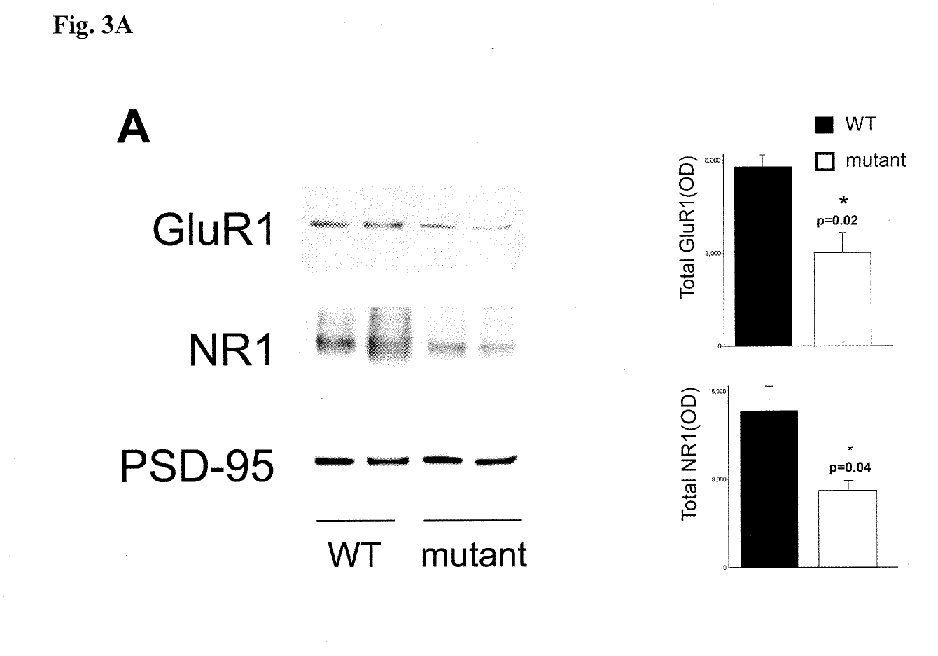

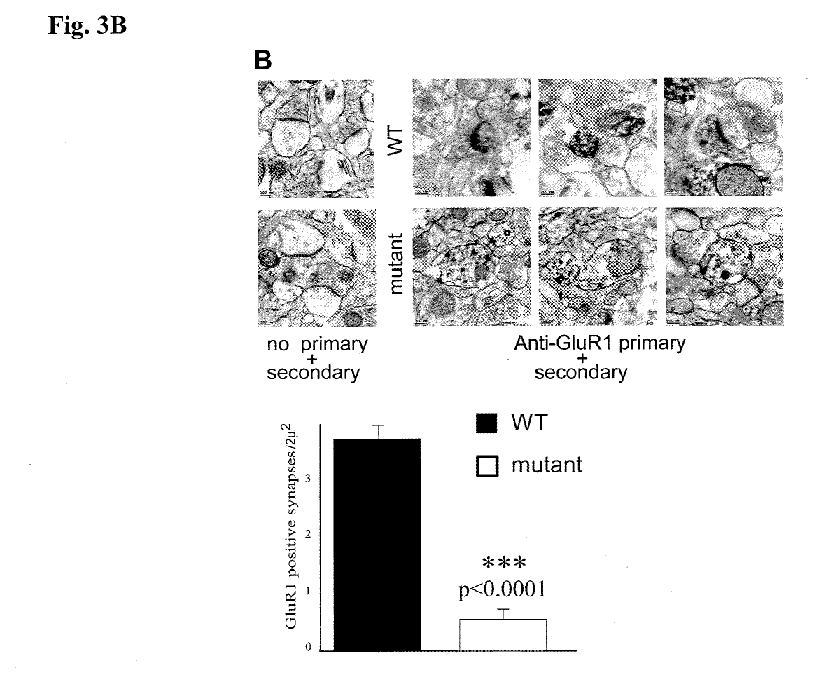

To determine whether the decreased NMDAR-mediated synaptic transmission is caused by a deficit in NR1 synaptic incorporation in the S897A mutant mice, we prepared synaptic membrane associated proteins by biochemical fractionation of hippocampal tissue dissected from either the wild type or the mutant mice. The mutant mice showed a significant decrease in NR1 protein level only in the synaptic fraction (FIG. 3A), whereas NR1 level in the total brain homogenate was unchanged (representative blot FIG. 1C). Moreover, GluR1 protein level in the synaptic faction (FIG. 3A), but not in the total homogenate (not shown), is also significantly reduced in the mutant animals, consistent with the impairment in AMPAR-mediated synaptic transmission (FIG. 2). GluR1-containing AMPAR synaptic insertion requires NMDAR activation (S. H. Shi et al., 1999), which underlies LTP in the Schafer collateral—CA1 synapses (R. Malinow and R. C. Malenka, 2002). We therefore also examined the localization of GluR1 in the dendritic spines in the CA1 area of the hippocampus by electron microscopy after immunostaining with a specific antibody against GluR1. Strikingly, unlike the wild type mice that showed strong GluR1 immunoreactivity in the postsynaptic density (PSD) region, mutant animals had markedly reduced GluR1 staining near the PSD regions and often formed abnormal clusters within the dendritic spines (FIG. 3B). Quantification of the total number of synapses that positively stained with GluR1 indicates a highly significant decrease in the number of GluR1 positive synapses in the mutant animals (p<0.0001 by t-test, histogram bar in FIG. 3B). These data suggest that preventing NR1 S897 phosphorylation caused severe impairment in synaptic NMDAR function such that it is not sufficient to drive effectively GluR1 into the synapse during synaptic plasticity, as evidenced by the reduction in basal synaptic transmission (FIG. 2A-C) and impairment in LTP in the mutant animals (FIG. 2 D).

Since the S897A mutation caused severe impairments in glutamatergic synaptic function and plasticity, we wondered whether it could also lead to behavioral deficits. To test this, we examined the mutant mice with behavioral assays commonly used to examine behavioral deficits in rodent models of schizophrenia. We first examined the animal's home cage motor activity and found that across all the parameters measured (horizontal/vertical activity and total distance traveled), there was no significant difference between the homozygous mutant animals and their wild-type littermates (data not shown), suggesting that the S897A mutation does not affect the overall motor function.

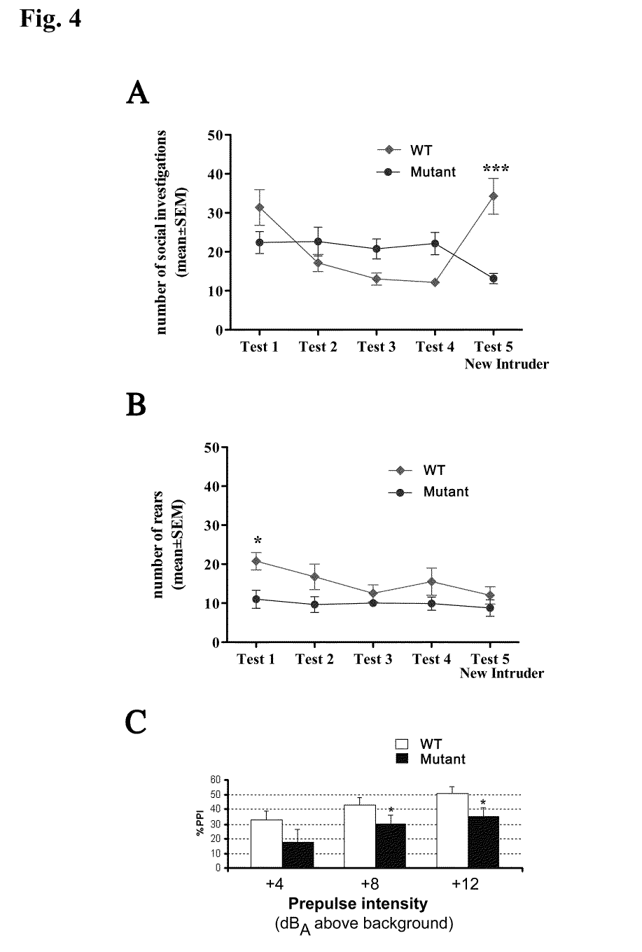

To test if the phosphomutant animals exhibit deficits in social interaction, we employed an assay that examines the social interest in rodents while minimizing the variability that can be introduced by different intruders in a typical social interaction assay based on intruder mice (E. Choleris et al., 2003). In this assay, each experimental mouse was tested five times (tests 1-5) for its reaction to a cylinder containing a stimulus mouse. In tests 1-4, the same stimulus mouse was used, whereas in test 5 a novel stimulus mouse was introduced. The behavioral data collected included social investigation (active sniffing of the holes near the bottom of the cylinder) and rearing (not in relation to the cylinder). The wild-type mice showed the expected decrease in social response (habituation to the stimulus mouse) throughout tests 1-4 (comparison between tests 1 vs. 4, P<0.001, N=8; FIG. 4A). The wild-type mice also showed the expected increase in social interest when presented with a novel animal at test 5 (comparison between test 4 vs. test 5, P<0.001, N=8; FIG. 4A). However, the mutant mice did not show the expected decline in the number of social investigations toward the repeatedly introduced stimulus mouse (comparison between tests 1 vs. 4, P>0.05, N=8; FIG. 4A), and also failed to show the expected increase in social investigation toward the novel stimulus mouse (comparison between tests 4 vs. 5, P>0.05, N=8; FIG. 4A). Analysis of the frequency of vertical activity (rearing) showed that the mutant mice exhibited significantly less number of rearing compared to their wild-type littermates in test 1 (FIG. 4B). However, in tests 2-5 the difference in vertical activity between mutant and wild-type mice was not significant (FIG. 4B). The difference in test 1 but not in tests 2-5 could be due to a difference between the wild-type and mutant mice in response to a novel environment. To test whether the deficit in social behavior of the NR1 S897A mutant animals could be explained by a change in olfactory function, we examined the mice for olfactory responsiveness and found that the mutant mice showed normal response to an olfactory stimulus, which was measured as the response to the odor of an 100% benzaldehyde solution presented when the mice were in resting state (see Methods section; data not shown). These results demonstrate that the NR1 S897A mutation impairs the function of the brain system that underlies normal social behaviors.

Schizophrenia patients show sensorimotor gating deficits that can be measured as an impairment in prepulse inhibition (PPI), in which a weak auditory prestimulus or prepulse reduces the startle response to a subsequent intense auditory stimulus (D. C. Javitt et al., 2008). Abnormal PPI has also been used as an indicator of impaired sensorimotor gating in rodent models of schizophrenia (N. R. Swerdlow and M. A. Geyer, 1998; M. A. Geyer et al., 2002). To determine whether the S897A mutation could affect sensorimotor gating, we examined PPI in the phosphomutant animals and their wild-type littermates. We used a combination of one startle stimulus (120 dB_{A}) preceded by one of the three prepulse stimuli of different intensities (+4, +8 and +14 dB_{A} above the background noise). As shown in FIG. 4C, the phosphomutant animals showed significantly decreased PPI for prepulse intensities of 8 or 12 dB_{A} above the background noise (at +8 dB_{A}: P<0.05, N=10; at +12 dB_{A}: P<0.05, N=10). We also evaluated the amplitude of the baseline acoustic startle response (in the absence of prepulses). There was no significant difference in the startle response amplitude between the mutant and wild-type animals. These results demonstrate that the NR1 S897A mutation impairs the plasticity of the startle response (i.e. PPI), while leaving the sensory responsiveness intact, and indicate that NR1 S897 phosphorylation plays a critical role in regulating the function of the neural circuitry underlying sensorimotor gating.

In this study we found that preventing NR1 S897 phosphorylation in vivo by substituting serine with alanine in mice severely reduces the level and function of NMDARs in the synapse, impairs synaptic plasticity and GluR1 synaptic incorporation, and impairs AMPAR-mediated synaptic transmission. Furthermore, the phosphomutant mice also show deficits in social activities and sensorimotor gating.

Earlier studies in cell cultures showed that overexpressed NR1 subunits are retained in the ER due to an ER retention signal near S897 (S. Standley et al., 2000), and mutations that mimic S897 phosphorylation can suppress the ER retention and facilitate NR1 exiting from ER, thereby regulating the level of NMDARs on cell surface (H. Xia et al., 2001; D. B. Scott et al., 2003). Therefore the impairment in NR1 synaptic incorporation in the phosphomutant animals could be due to a failure in phosphorylating NR1 S897 and increased NR1 ER retention. However, ER retention of wild type NR1 is relieved when it is co-expressed with NR2 subunits (A. Barria and R. Malinow, 2002). Since mutant animals express NR1 (FIG. 1D) and NR2 subunits (not shown) at levels similar as wild type animals, the amount of fully assembled NMDARs that can exit ER in the mutant animals may be comparable to that of wild-type animals. Therefore the reduction of synaptic NMDARs in the NR1 phosphomutant animals could be due to an impairment downstream of ER retention, such as faulty NMDAR synaptic incorporation.

The reduction of AMPA currents and AMPAR synaptic incorporation in the mutant animals could be caused by the primary impairment in NMDAR-mediated synaptic transmission, since NMDARs are essential for both synaptic maturation and plasticity (M. Constantine-Paton and H. T. Cline, 1998). Interestingly, recent studies showed that complete deletion of NR1 (H. Adesnik et al., 2008; D. Engblom et al., 2008; L. S. Zweifel et al., 2008) or NR2B (B. J. Hall et al., 2007) results in potentiation of AMPAR-mediated synaptic transmission. It is possible that low levels of NMDAR function during development act to reduce synaptic AMPAR levels (M. Sheng and M. J. Kim, 2002). The NR1 S897 mutation, which produces low levels of NMDAR function, could enhance this effect. The complete loss of NMDAR function during early development, as achieved by genetic deletion of either NR1 (H. Adesnik et al., 2008; D. Engblom et al., 2008; L. S. Zweifel et al., 2008) or NR2B (B. J. Hall et al., 2007), prevents this normally occurring reduction, leading to a net potentiation of AMPAR transmission. Alternatively, changes in signaling pathways that are normally coupled to NMDAR via NR1 phosphorylation could account for the synaptic depression in the NR1 S897A mutant animals. Further studies are needed to clarify these issues.

Social behavior and sensorimotor gating are impaired in both schizophrenia patients and rodent models of schizophrenia. In this study, we found that the NR1 S897A mutant mice display significant impairments in social behaviors and sensorimotor gating, as measured by a social interaction paradigm and PPI, respectively. Notably, social abnormalities and PPI deficit similar to those that we observed in the NR1 S897 phosphomutant mice can also be induced in rodents by NMDAR antagonists such as phencyclidine, MK801, and ketamine (N. R. Swerdlow and M. A. Geyer, 1998; B. A. Ellenbroek and A. R. Cools, 2000; M. A. Geyer et al., 2001; M. A. Geyer et al., 2002; C. M. Powell and T. Miyakawa, 2006). However, neither of the two behavioral phenotypes we observed in the mutant mice is specific to schizophrenia. For example, social interaction deficits can be seen in autism (S. S. Moy and J. J. Nadler, 2008), and PPI deficits can be seen in other disorders as well (S. G. Giakoumaki et al., 2007). Furthermore, it is difficult to draw an analogy between rodent social behaviors, which are primarily driven by olfactory function, with those of humans. Further studies, including a comprehensive battery of behavioral tests that examine different aspects of cognitive function, are necessary to determine if this mutation can cause specific cognitive deficits in rodents that are related to schizophrenia or other psychiatric or neurological disorders.

There are several scenarios that can explain how a decrease in NR1 S897 phosphorylation could contribute to abnormal behavior. A decrease in NMDAR function, such as that caused by genetic deficits (H. Stefansson et al., 2002; B. Li et al., 2007) or pharmacological treatments (D. D. Xu et al., 2006; A. Mouri et al., 2007; G. L. Snyder et al., 2007), may lead to inefficient phosphorylation of NR1 at S897, which further impairs NMDAR function, resulting in a positive-feedback like mechanism. This will result in impairments in synaptic function and plasticity, and abnormal behaviors. Alternatively, since NR1 S897 is phosphorylated by PKA (W. G. Tingley et al., 1997), a primary change in PKA or a phosphotase (such as calcineurin) activity could also lead to changes in S897 phosphorylation. Interestingly, both PKA pathway and calcineurin have been linked to schizophrenia (A. Sawa and S. H. Snyder, 2005; Y. Horiuchi et al., 2007; Y. Kinoshita et al., 2008).

The findings of our study provide new insight for the mechanisms by which NMDAR hypofunction could occur. The NR1 S897A mice therefore can serve as a genetic tool for further circuit and behavioral analysis.

Applications

The present invention is further directed to a method for the evaluation of the in vivo effects of compounds on glutamatergic function through the use of the novel mutant animals provided by the invention. Applicants have generated mutant mice expressing a modified version of, e.g., the NR1 gene to elucidate the in vivo mechanism of synaptic plasticity. Expression of the modified gene is confirmed by PCR analysis or by assaying for mutant protein that is detectable, e.g., by RPA, Western blot or like analysis.

In an embodiment of this aspect, the invention provides a method of screening for compounds capable of modulating glutamatergic function, comprising providing a mutant animal having a modified NMDA Receptor, wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1; administering an effective amount of a test agent to the mutant animal and a control animal; measuring for a change in at least one of NMDAR activity, NR1 gene expression, NR1 and/or NR2 phosphorylation state or level in the mutant animal and the control animal; wherein, a change in at least one of NMDAR activity, NR1 gene expression, NR1 and/or NR2 phosphorylation state or level is indicative of an agent that modulates glutamatergic function. In one embodiment, the animal is a mouse. In certain embodiments, the amino acid substitution is at Serine 897. In an additional embodiment, Serine 897 is substituted with Alanine. In certain additional embodiments, Serine 897 is substituted with Glutamate or Aspartate. In an additional embodiment, the agent is an agonist of at least one of NMDAR activity, NR1 gene expression, NR1 and/or NR2 phosphorylation state or level. In still another embodiment, the agent is an antagonist of at least one of NMDAR activity, NR1 gene expression, NR1 and/or NR2 phosphorylation state or level.

In another aspect, the invention provides a method of screening for compounds for treating psychiatric and/or neurological disorders, comprising providing a mutant animal having a modified NMDA Receptor, wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1; administering an effective amount of a test agent to the mutant animal and a control animal; measuring for a change in at least one of NMDAR activity, NR1 gene expression, NR1 and/or NR2 phosphorylation state or level, the cognitive ability or behavior in the mutant animal and the control animal; wherein, a change in at least one of NMDAR activity, NR1 gene expression, NR1 and/or NR2 phosphorylation state or level, the cognitive ability or behavior is indicative of an agent useful for treating at least one of cognitive disorders, neurological disorders, and/or behavioral disorders. In one embodiment, the animal is a mouse. In certain embodiments, the amino acid substitution is at Serine 897. In an additional embodiment, Serine 897 is substituted with Alanine. In certain additional embodiments, Serine 897 is substituted with Glutamate or Aspartate. In an additional embodiment, the agent is an agonist of at least one of NMDAR activity, NR1 gene expression, NR1 and/or NR2 phosphorylation state or level. In still another embodiment, the agent is an antagonist of at least one of NMDAR activity, NR1 gene expression, NR1 and/or NR2 phosphorylation state or level. In certain embodiments, the psychiatric and/or neurological disorders are selected from the group consisting of cognitive disorders, behavioral disorders, psychotic disorders such as schizophrenia and other and delusional disorders, autism, Alzheimer's Disease, ischemic brain disorders such as stroke, dementia, anesthesia and cognitive dysfunction associated with it, amnesia, delirium, anxiety disorders such as phobias, panic disorders, obsessive-compulsive disorder, generalized anxiety disorder, post-traumatic stress disorder, and mood disorders such as depression and bipolar disorder.

Potential NMDA receptor antagonists include: Amantadine, Ketamine, Phencyclidine (PCP), Nitrous oxide, Dextromethorphan, and dextrorphan, Memantine, Ethanol, Riluzole (used in ALS), Xenon, HU-211 (also a cannabinoid), Lead (Pb2+), Dual opioids and NMDA-Antagonists: Ketobemidone, Methadone, Dextropropoxyphene, Tramadol, Kratom alkaloids, Ibogaine.

The NMDA receptor is modulated by a number of endogenous and exogenous compounds: Mg²⁺ not only blocks the NMDA channel in a voltage-dependent manner but also potentiates NMDA-induced responses at positive membrane potentials. Magnesium glycinate and magnesium taurinate treatment has been used to produce rapid recovery from depression; Na⁺, K⁺ and Ca²⁺ not only pass through the NMDA receptor channel but also modulate the activity of NMDA receptors; Zn²⁺ blocks the NMDA current in a noncompetitive and a voltage-independent manner; Pb2+ lead is a potent NMDAR antagonist. Presynaptic deficits resulting from Pb2+ exposure during synaptogenesis are mediated by disruption of NMDAR-dependent BDNF signaling; It has been demonstrated that polyamines do not directly activate NMDA receptors, but instead act to potentiate or inhibit glutamate-mediated responses; Aminoglycosides have been shown to have a similar effect to polyamines, and this may explain their neurotoxic effect; The activity of NMDA receptors is also strikingly sensitive to the changes in H⁺ concentration, and partially inhibited by the ambient concentration of H⁺ under physiological conditions. The level of inhibition by H⁺ is greatly reduced in receptors containing the NR1a subtype, which contains the positively-charged insert Exon 5. The effect of this insert may be mimicked by positively-charged polyamines and aminoglycosides, explaining their mode of action.

In additional aspects the invention relates to compositions and methods related to the treatment of neurological and/or psychiatric pathologies and conditions, including schizophrenia. In certain exemplary embodiments, the invention encompasses, for example, the administration of an effective amount of a therapeutic composition of the invention to an individual for the treatment and/or prevention of schizophrenia. In an embodiment, the invention provides a method of treating diabetes comprising administering to an individual a composition comprising an effective amount of an agent that performs at least one of: increasing the expression of the NR1 gene, increasing the activity of NMDAR polypeptide, or a combination of both, wherein the agent is effective for the treatment of a disorder, e.g., a psychiatric disorder. In certain embodiments, the composition is administered systemically. In any of the methods described herein, the nucleic acids or polypeptides of the invention may be delivered or administered in any pharmaceutically acceptable form, and in any pharmaceutically acceptable route as described in further detail below. For example, compositions comprising nucleic acids and/or polypeptides of the invention can be delivered systemically or administered directly to a cell or tissue. In certain additional embodiments, the nucleic acids and/or polypeptides of the invention comprise a carrier moiety that improves bioavailability, increases the drug half-life, targets the therapeutic to a particular cell or tissue type, for example, skeletal or striated muscle cells or tissues, or combinations thereof.

In yet another aspect, the invention provides a method for determining the presence of or predisposition to a disease associated with a neurological pathology or psychiatric disease in a subject (e.g., a human subject). In one embodiment, the method comprises isolating a biological sample from an individual (e.g., blood, brain, or other), detecting the genotype of an NR1 gene by treating a tissue sample from an individual with a detectable probe specific for an NR1 polymorphism or mutation, and detecting the formation of a probe/target complex, wherein formation of a complex is indicative of the presence of a particular genotype. In another embodiment, the method comprises steps for diagnosing or monitoring disorder or disease or progression comprising isolating a biological sample from an individual, detecting for the presence of a nucleotide polymorphism in an NR1 gene as described herein, wherein the NR1 polymorphism is associated with the disease or its severity.

In an embodiment, the invention comprises a method for screening for agents that modulate at least one of NMDAR activity, NR1 and/or NR2 subunit protein levels, or gene expression (i.e., an NMDAR agonist and/or antagonist) comprising providing mutant animal provided by the invention, or a cell or tissue from the same; measuring for the amount of at least one of endogenous NMDAR activity, protein level, or gene expression to establish a control value; contacting a test agent to the animal, cell or tissue; measuring or detecting the activity of at least one of NMDAR, amount of NR1 and/or NR2 protein, or amount of NR1 and/or NR2 gene expression to establish a test value; and comparing the control value to the test value, wherein an observed change between the test and control values indicates an agent capable of modulating at least one of NMDAR activity, NR1 and/or NR2 protein levels, or NR1 and/or NR2 gene expression in the cell or tissue.

Binding of the test compound to NR1 and/or NR2 nucleic acid or polypeptides indicates the test compound is a modulator of activity, transcription, translation or of latency or predisposition to the aforementioned disorders or syndromes. In another embodiment, the invention provides a method for screening for agents that modulate behavior comprising contacting a mutant animal provided by the invention that expresses a modified NR1 and/or NR2 gene, with an agent that modulates the expression of NR1 and/or NR2, activity of NMDAR or a combination thereof, and measuring the effects on the animal's behavior versus a control or non-mutant animal, wherein a change in behavior is indicative of an NMDAR agonist or antagonist.

Libraries of potential compounds are widely known and readily available that could be used in the methods of the invention. Additional methods useful for practicing the invention are routinely used and can be adapted for use in the claimed methods using routine experimentation for the art.

Certain aspects of the invention encompass methods of detecting gene expression or polymorphisms with one or more DNA molecules wherein the one or more DNA molecules has a nucleotide sequence which detects expression of a gene corresponding to the oligonucleotides depicted in the Sequence Listing (See TABLES 1 and 2). In one format, the oligonucleotide detects expression of a gene that is differentially expressed. The gene expression system may be a candidate library, a diagnostic agent, a diagnostic oligonucleotide set or a diagnostic probe set. The DNA molecules may be genomic DNA, RNA, protein nucleic acid (PNA), cDNA or synthetic oligonucleotides. Following the procedures taught herein, one can identify sequences of interest for analyzing gene expression or polymorphisms. Such sequences may be predictive of a disease state. Polymorphisms have been identified that correlate with disease severity. (See, Zhong et al., Simultaneous detection of microsatellite repeats and SNPs in the macrophage migration inhibitory factor gene by thin-film biosensor chips and application to rural field studies. Nucleic Acids Res. 2005 Aug. 2; 33(13):e121; Donn et al., A functional promoter haplotype of macrophage migration inhibitory factor is linked and associated with juvenile idiopathic arthritis. Arthritis Rheum. 2004 May; 50(5):1604-10; all of which are incorporated herein by reference in their entirety for all purposes.). As one of ordinary skill will comprehend, polymorphisms associated with any of the disorders indicated herein, and hence useful as diagnostic markers according to the methods of the invention, may appear in any of the nucleic acid regions of the NR1 or NR2 gene or regulatory regions. Techniques for the identification and monitoring of polymorphisms are known in the art and are discussed in detail in U.S. Pat. No. 6,905,827 to Wohlgemuth, which is incorporated herein by reference in its entirety for all purposes.

Host Cells

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as primates, humans, cows, sheep, apes, monkeys, swine, dogs, mice, rats, and cats. The cell can be prokaryotic (e.g., bacterial cell) or eukaryotic (e.g., mammalian or plant cell). The term “host cell” includes a cell that might be used to carry a heterologous or exogenous nucleic acid, or expresses a peptide or protein encoded by a heterologous or exogenous (i.e., foreign) nucleic acid. A host cell can contain genes that are not found within the native (non-transformed) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the invention.

Transformation of a host cell with recombinant DNA may be carried out by conventional techniques as are well known to those skilled in the art. By “transformation” is meant a permanent or transient genetic change induced in a cell following introduction, modification, and/or extraction of nucleic acid material, for example, DNA or RNA.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. These examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

In another aspect, the invention encompasses a host cell comprising any NR1 and/or NR2 nucleic acid of the invention. In certain embodiments, the host cell comprises a vector that contains a recombinant NR1 and/or NR2 nucleic acid; or a nucleic acid complementary to an NR1 and/or NR2 encoding nucleic acid; or an exogenous or recombinant promoter modulating expression of endogenous NR1 and/or NR2 gene.

Formulations

In any of the embodiments described herein, a therapeutic provided by the invention can be administered together with a pharmaceutically acceptable carrier, excipients, and/or an adjuvant. In additional embodiments, the invention provides therapeutic composition comprising a composition provided by the invention in combination with at least one additional biologically active and/or therapeutic agent such as an amino acid, peptide, polypeptide, chemical compound, drug, antibody or the like, or a combination thereof. For example, in an embodiment the therapeutic composition comprises an NR1 and/or NR2 nucleic acid and/or NR1 and/or NR2 polypeptide in combination with at least one additional biologically active and/or therapeutic agent such as an amino acid, peptide, polypeptide, chemical compound, drug, antibody or the like, or a combination thereof. The invention also provides methods of administering the same for the treatment or amelioration of a muscle related condition, including diabetes.

In any aspect of the invention, the nucleic acid or polypeptide compositions of the invention can be in any pharmaceutically acceptable form and administered by any pharmaceutically acceptable route, for example, the therapeutic composition can be administered as an oral dosage, either single daily dose or unitary dosage form, for the treatment of a muscle disorder or conditions, e.g., diabetes. Such pharmaceutically acceptable carriers and excipients and methods of administration will be readily apparent to those of skill in the art, and include compositions and methods as described in the USP-NF 2008 (United States Pharmacopeia/National Formulary), which is incorporated herein by reference in its entirety. In certain aspects, the invention provides pharmaceutically acceptable formulations of the compounds described. These formulations include salts of the above compounds, e.g., acid addition salts, for example, salts of hydrochloric, hydrobromic, acetic acid, and benzene sulfonic acid.

The active compounds will generally be formulated for parenteral administration, e.g., formulated for injection via the intravenous, intraarthricular, intrathecal, intramuscular, sub-cutaneous, intra-lesional, or even intraperitoneal routes. The preparation of an aqueous composition that contains a cancer marker antibody, conjugate, inhibitor or other agent as an active component or ingredient will be known to those of skill in the art in light of the present disclosure. Typically, such compositions can be prepared as injectibles, either as liquid solutions or suspensions; solid forms suitable for using to prepare solutions or suspensions upon the addition of a liquid prior to injection can also be prepared; and the preparations can also be emulsified.

A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The compounds, nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The formulations can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release of the active compound. For buccal administration the compositions may take the form of tablets or lozenges formulated in conventional manner.

Excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present. Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for injectable use include sterile aqueous solutions (where water soluble) or dispersions and sterile powders for the extemporaneous preparation of sterile injectable solutions or dispersion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. For intravenous administration, suitable carriers include physiological saline, bacteriostatic water, Cremophor™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

For administration to non-human animals, the therapeutic compositions of the invention can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water. The composition can also be administered to a subject in combination with other therapeutic compounds to increase the overall therapeutic effect. The use of multiple compounds to treat an indication can increase the beneficial effects while reducing the presence of side effects.

Exemplary Methods

Generation and characterization of S897A NR1 phosphomutant mice. To construct the targeting vector, a DNA fragment containing the C-terminus of NR1 was isolated from the BAC library. Using a PCR strategy the amino acid serine at position 897 was mutated to an alanine A LoxP-FRT-Neo-FRT cassette was inserted into the intron between Exon 18 and Exon 19 of NR1 and correct orientation was confirmed by sequencing. Linearized targeting vector was injected into ES cells. In the recombinant ES cells the FRT-Neo-FRT cassette was excised using FLP recombinase. Positive ES cell clones were injected into C57BL/6 blastocysts. The resulting chimeras were crossed with C57BL/6 mice. Heterozygous mice were bred to produce homozygotes and wild-types. Successful gene targeting was confirmed by sequencing the genomic DNA from the mutant mice. Using a specific antibody against the phosphorylated NR1 at S897, the deficiency of NR1 S897 phosphorylation in the mutant mice was confirmed by western blot analysis (described at below).

Western blot analysis. Tissues from frontal cortex, striatum, or hippocampus of mouse brain (0.05-0.1 gr) were homogenized in ice-cold lysate buffer (0.25 M Tris, pH 7.5) containing protease inhibitors (Protease Inhibitor Cocktail tablets, Boehringer Mannheim) and phosphatase inhibitors (Phosphatase Inhibitor Cocktails I & II, Sigma), and were lysed through three cycles of freezing (in liquid nitrogen) and thaw (in 37° C. water bath). Protein concentration was measured (Bio-Rad protein assay) with spectrometry at 595 nm. Equal amounts of total protein were loaded on 4-12% gradient Bis-Tris gels, separated using the NuPAGE system (Invitrogen) and transferred onto nitrocellulose membrane. The membrane was probed with primary and secondary antibodies and signals were detected by chemilluminescence followed by autoradiography. The following antibodies were used: anti-NR1 (raised against a conserved sequence of different NR1 splice variants, BD PharMingen and Upstate Biotechnology, 1:1000), anti-GluR1 (Chemicon, 1:1000), anti-S897 NR1 (Cell Signaling, 1:500 and Upstate Biotechnology, 1:5000), and anti-PSD-95 (Upstate Biotechnology, 1:2000).

Immunohistochemistry and electron microscopy. Mice were anaesthetized and perfused with 4% paraformaldehyde, and brain tissues were removed and post fixed overnight. Parasagital brain sections of 60 μm thickness were obtained using a vibratome and were briefly treated with 0.1% glutaraldehyde. Sections were then washed with 0.1 M phosphate buffer, pH 7.4, and were blocked overnight with 2% normal goat serum. Sections were incubated for 48 h in blocking solution with 1:400 dilution of anti-GluR1 antibody (Chemicon). Sections were washed four times, 15 min each, in 0.1 M tris-saline buffer and incubated with anti-rabbit secondary antibody (included in the ABC kit, Vector) for 16 h, and were then washed four times, 15 min each, in tris-saline buffer. Using the ABC kit (Vector) the brain sections were then subjected to the DAB reaction following the kit instructions. Electron microscopy analysis was performed following the methods of Brown and Farquhar (Methods Cell Biol. 31, 553-569, 1989). Sections were post-fixed and stained in reduced osmium tetroxide (1% OsO4, 1% potassium ferrocyanide, in 0.1 m cacodylate) for 1 hour on ice, and were dehydrated through a graded series of ethanol (50%, 70%, 95% and 100%). Following this step, samples were treated with 100% propylene oxide for 30 minutes and infiltrated with Epon:propylene oxide(1:1) overnight. Samples were treated with two changes of 100% Epon, 2 hours each. Sections were then cured on top of pre-cured blocks in a 60° C. oven for 48 hours. Ultra thin sections were cut with a diamond knife on Ultracut-E (Reichert-Jung) and post-stained with Uranyl acetate and Lead. Sections were scanned and pictures were taken on a Tecnai Transmission Electron microscope (FEI) fitted with Gatan 895 UltraScan Digital Camera.

Preparation of synaptic associated proteins. Hippocampi of both brain hemispheres were dissected and homogenized in 0.5 ml of 0.32M sucrose supplemented with protease and phosphatase inhibitors. 50 •l of each sample was stored, and another 1 ml of sucrose was added to the rest of each sample. Samples were centrifuged at 800×g for 10 minutes. Supernatants were collected and spun at 10,000×g for another 10 minutes. The supernatants were stored and the pellets were re-suspended in a buffer (containing 0.5% Triton X-100, 120 mM NaCl, 50 mM Tris pH. 7.4, 1 mM EDTA plus protease and phosphatase inhibitors). Samples were incubated on ice for 30 minutes and spun at 21000×g for 2 hours to isolate the PSD-95 fraction. Each fractionation experiment was tested by western blot analysis of equal amount of proteins from each fraction and probing with anti PSD-95 antibody.

Preparation of acute brain slices. Male, 22 to 25-days-old mice were used for all the electrophysiology experiments. Animals were anesthetized with isoflurane, decapitated and the brains quickly removed and chilled in ice-cold dissection buffer (110.0 mM choline chloride, 25.0 mM NaHCO3, 1.25 mM NaH2PO4, 2.5 mM KCl, 0.5 mM CaCl2, 7.0 mM MgCl2, 25.0 mM glucose, 11.6 mM ascorbic acid, 3.1 mM pyruvic acid; gassed with 95% O2/5% CO2). Coronal slices (300 μm) were cut in dissection buffer using a VT-1000 S vibratome (Leica, Nussloch, Germany) and subsequently transferred to a storage chamber containing artificial cerebrospinal fluid (ACSF; 118 mM NaCl, 2.5 mM KCl, 26.2 mM NaHCO3, 1 mM NaH2PO4, 20 mM Glucose, 4 mM MgCl2, 4 mM CaCl2; 22°-25° C.; pH 7.4; gassed with 95% O2/5% CO2). After at least 1 hour of recovery time slices were transferred to the recording chamber and were constantly perfused with ACSF maintained at 27° C.

Electrophysiology. Experiments were always performed on interleaved mutant and littermate wild-type animals for comparison. About ⅔ of the experiments were done blind, which showed the same results and were combined with the rest of the data. Whole-cell recordings were obtained with Axopatch-1D amplifiers (Axon Instruments) onto pyramidal neurons in the CA1 region of hippocampus under visual guidance using transmitted light illumination. Synaptic transmission was evoked with a bipolar stimulating electrode placed in the Schaffer collateral pathway, ˜0.2 mm away from cell bodies. Responses were recorded at holding potentials of both •60 (for AMPA receptor-mediated responses) and +40 (for NMDAR-mediated responses) mV. NMDA receptor-mediated responses were quantified as the mean between 110 and 160 ms after stimulation. Bathing solution (ACSF) contained (in mM): 119 NaCl, 2.5 KCl, 4 CaCl₂, 4 MgCl₂, 26.2 NaHCO₃, 1 NaH₂PO₄, 11 glucose, 0.1 picrotoxin and gassed with 5% CO₂ and 95% O₂ at 27° C. Internal solution contained (in mM): 115 cesium methanesulfonate, 20 CsCl, 10 HEPES, 2.5 MgCl₂, 4 Na₂-ATP, 0.4 Na-GTP, 10 Na-phosphocreatine, and 0.6 EGTA (pH 7.2). Spontaneous responses (mEPSCs) were recorded at 27° C. in the presence of 1 •M TTX and 0.1 mM picrotoxin and analyzed using Mini Analysis Program (Synaptosoft). For recordings on mEPSC or field EPSPs, the concentration for Ca²⁺ and Mg²⁺ were adjusted to 2.5 and 1.3 mM, respectively. Electrodes (˜1 m•) were filled with 1 M NaCl and placed in the dendritic area (˜0.25 mm from the somas in CA1) to record field EPSPs. LTP was induced by stimulating the Schaffer—collateral pathway (two trains of 1 second pulses at 100 Hz, at an interval of 25 seconds). The result of the LTP experiments was displayed as EPSP amplitude normalized to the average of the responses before LTP induction.

Animals for the behavioral assays. A total of 8 male homozygous phosphomutant mice and 8 of their wild-type littermates were tested for social interaction, and 10 pairs of homozygous mutants and their wild-type littermates were studied for prepulse inhibition. Adult (6-12 weeks) male cohorts of paired homozygous and their wild-type littermates (with similar body weight) were used for the behavioral assays of this study. Mice were housed individually and were maintained on a 12/12 light/dark cycle with light on at 18:00. All animals had food and water available ad libitum and were cared for in accordance with the Rockefeller University Animals Care and Use Committee (IACUC) protocol.

Social interaction. The social interaction paradigm was employed as previously described (E. Choleris et al., 2003). Briefly, a stimulus mouse was presented to an experimental mouse in its home cage, which was covered with a clear Plexiglas top (23×33 cm). The stimulus mouse was placed in a clear Plexiglas cylinder (9 cm in diameter, 10 cm in height) with 20 holes (4 mm diameter) drilled near the bottom of the cylinder to allow the passage of olfactory cues while preventing direct interaction between the experimental and the stimulus mouse. All testes were done at the dark phase of the light/dark regime. Before testing, all mice were moved to the darkened testing room with a small red light, and experimental mice were habituated to the presence of an empty cylinder in their home cage for 10 min. Similarly, the stimulus mice were also placed in the clear cylinders and habituated for 5 min prior to testing. Each experimental mouse was tested five times (tests 1-5) in their home cage, where a cylinder containing a stimulus mouse was introduced. Each test lasted 5 min, with 15 min intervals in between. In tests 1-4, the same stimulus mouse was used, whereas in test 5, a novel stimulus mouse was used. During testing, mice were left undisturbed in the room and their behavior was videotaped (8-mm Cannon Handycam) for subsequent analysis. During the 15-min inter-test intervals, the same empty cylinder was placed back in the cage. The position of all the cylinders introduced into the mouse home cage was kept constant throughout the experiment. After every use, cylinders were thoroughly washed with unscented soap and then dried by paper towel. The behavioral data collected includes social investigation (active sniffing of the holes) and rearing (not in relation to the cylinder).

Animals' olfactory function was examined to make sure that the deficit in social behavior of the NR1 S897A mutant animals was not caused by changes in the olfactory system. An olfactory stimulus, which was the odor from 100% benzaldehyde solution (Sigma, St. Louis, Mo.), was presented for a 20 sec duration when mice were in a “resting state”, i.e. there was no home cage activity detected by the computer (i.e., the Total Distance (TD) traveled, Horizontal Activity (HA) and Vertical Activity (VA) were zero) for at least 5 min. The changes in the animals' home cage activity (TD, HA and VA) were measured until the animals reached the resting state again (approximately 10 min).

Prepulse inhibition (PPI) of the acoustic startle response. Prepulse inhibition (PPI) of the acoustic startle response was measured as described previously with some minor modifications (E. S. Emamian et al., 2004b). Adult homozygous mutant mice and their wild-type littermates were housed individually for 2 weeks prior to testing. Testing was conducted in a SR-Lab system (San Diego Instruments). Response amplitude was calculated as the maximum response level occurred during the 100 ms recording. Because animals can habituate to the prepulse, and to the startle stimulus, the number of trials was kept to minimum. Immediately after being placed in the chamber, the animal was allowed to acclimate for 5 min which background noise (86 dB) continually present. The animal then received 10 sets of the following 5 types of trials, distributed pseudo-randomly, and separated by 15 sec inter-trial intervals: Trial 1: 40 ms, 120 dB noise burst alone; Trial 2-4: 120 dB startle stimuli preceded 100 ms by one of the three 20 ms prepulses: 90 dB, 94 dB or 98 dB; Trial 5: no-stimulus/background noise alone (86 dB). As a control experiment to examine the efficacy of PPI protocol used in this study, wild type C57BL6 mice were injected with MK-801 (1 mg/kg, using the methods of (B. K. Yee et al., 2004)) and subjected to the same PPI protocol described here. This control experiment showed that injection of MK-801 resulted in a significant decrease in PPI (data not shown) using this PPI protocol. Data was analyzed using ANOVA with repeated measures.

Those skilled in the art will recognize, or be able to ascertain using no more than routine experimentation, many equivalents to the specific embodiments of the invention described herein. Such equivalents are intended to be encompassed by the following claims.

What is claimed is:

1. A mutant animal having a modified NMDA Receptor (NMDAR), wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1.

2. The mutant animal of claim 1, wherein the animal is a mouse.

3. The mutant animal of claim 2, wherein the amino acid substitution is at Serine 897.

4. The mutant animal of claim 3, wherein Serine 897 is substituted with Alanine.

5. The mutant animal of claim 3, wherein Serine 897 is substituted with Glutamate or Aspartate.

6. A method of screening for compounds capable of modulating glutamatergic function, comprising providing a mutant animal having a modified NMDA Receptor, wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1; administering an effective amount of a test agent to the mutant animal and a control animal; measuring for a change in at least one of NMDAR activity, NR1 gene expression, NR1 and/or NR2 phosphorylation state or level in the mutant animal and the control animal; wherein, a change in at least one of NMDAR activity, NR1 gene expression, NR1 and/or NR2 phosphorylation state or level is indicative of an agent that modulates glutamatergic function.

7. The mutant animal of claim 6, wherein the animal is a mouse.

8. The mutant animal of claim 7, wherein the amino acid substitution is at Serine 897.

9. The mutant animal of claim 8, wherein Serine 897 is substituted with Alanine.

10. The mutant animal of claim 8, wherein Serine 897 is substituted with Glutamate or Aspartate.

11. The method of claim 6, wherein the agent is an agonist of at least one of NMDAR activity, NR1 gene expression, NR1 and/or NR2 phosphorylation state or level.

12. The method of claim 6, wherein the agent is an antagonist of at least one of NMDAR activity, NR1 gene expression, NR1 and/or NR2 phosphorylation state or level.

13. A method of screening for compounds for treating psychiatric and/or neurological disorders, comprising providing a mutant animal having a modified NMDA Receptor, wherein the genome of the mutant animal comprises a genetic sequence alteration in the endogenous NR1 subunit gene such that it encodes an NR1 protein comprising an amino acid substitution of at least one of Serine 897, Serine 896, or Serine 890 of SEQ ID NO: 1; administering an effective amount of a test agent to the mutant animal and a control animal; measuring for a change in at least one of NMDAR activity, NR1 gene expression, NR1 and/or NR2 phosphorylation state or level, the cognitive ability or behavior in the mutant animal and the control animal; wherein, a change in at least one of NMDAR activity, NR1 gene expression, NR1 and/or NR2 phosphorylation state or level, the cognitive ability or behavior is indicative of an agent useful for treating at least one of cognitive disorders, neurological disorders, and/or behavioral disorders.

14. The mutant animal of claim 13, wherein the animal is a mouse.

15. The mutant animal of claim 14, wherein the amino acid substitution is at Serine 897.

16. The mutant animal of claim 15, wherein Serine 897 is substituted with Alanine.

17. The mutant animal of claim 15, wherein Serine 897 is substituted with Glutamate or Aspartate.

18. The method of claim 13, wherein the agent is an agonist of at least one of NMDAR activity, NR1 gene expression, NR1 and/or NR2 phosphorylation state or level.

19. The method of claim 13, wherein the agent is an antagonist of at least one of NMDAR activity, NR1 gene expression, NR1 and/or NR2 phosphorylation state or level.

20. The method of claim 13, wherein the psychiatric and/or neurological disorder is selected from the group consisting of cognitive disorders, behavioral disorders, psychotic disorders such as schizophrenia and other and delusional disorders, autism, Alzheimer's Disease, ischemic brain disorders such as stroke, dementia, anesthesia and cognitive dysfunction associated with it, amnesia, delirium, anxiety disorders such as phobias, panic disorders, obsessive-compulsive disorder, generalized anxiety disorder, post-traumatic stress disorder, and mood disorders such as depression and bipolar disorder.
